(19) United States
(12) Patent Application Publication
(10) Pub. No.: US 2022/0005560 A1
(43) Pub. Date: Jan. 6, 2022

(54) DATA GENERATION DEVICE, METHOD, AND NON-TRANSITORY STORAGE MEDIUM STORING PROGRAM

(71) Applicant: OMRON HEALTHCARE Co., Ltd., Kyoto (JP)

(72) Inventors: Daiki ADACHI, Kyoto (JP); Hiroshi OGAWA, Kyoto (JP); Mizuki SHIMOGAMA, Kyoto (JP)

(21) Appl. No.: 17/447,765

(22) Filed: Sep. 15, 2021

Related U.S. Application Data
(63) Continuation of application No. PCT/JP2020/013496, filed on Mar. 25, 2020.

(30) Foreign Application Priority Data
Mar. 25, 2019 (JP) 2019-057243

(51) Int. Cl. G16H 10/60; G16H 40/20; G06K 19/06; G16H 40/67; G16H 50/20; G16H 50/30; G06F 21/62; H04W 4/80; A61B 5/00

(52) U.S. Cl. CPC G16H 10/60; G16H 40/20; G06K 19/06037; G16H 40/67; A61B 5/7465; G16H 50/30; G06F 21/6245; H04W 4/80; G16H 50/20

(57) ABSTRACT

In a case where biometric data indicates an anomaly of a user, data corresponding to activities that the user and the doctor respectively should perform is generated based on the same biometric data. A data generation device includes a biometric data acquisition unit configured to acquire biometric data of a user, a generation unit configured to generate, based on the biometric data, user-oriented data directed to the user, and medical professional assistance data directed to a medical professional, the medical professional assistance data assisting an activity performed by the medical professional in relation to the user, and an access control unit configured to control knowability about the medical professional assistance data.

### CROSS-REFERENCE TO RELATED APPLICATIONS

This application is the U.S. national stage application filed pursuant to 35 U.S.C. 365(c) and 120 as a continuation of International Patent Application No. PCT/JP2020/013496, filed Mar. 25, 2020, which application claims priority to Japanese Patent Application No. 2019-057243, filed Mar. 25, 2019, which applications are incorporated herein by reference in their entireties.

### TECHNICAL FIELD

The invention relates to a data generation device, a method and a non-transitory storage medium storing a program.

### BACKGROUND ART

Recently, with improved performance of home-use medical measurement apparatuses, it has become possible for a user to measure various biometric data of the user with a high accuracy, acquire and easily refer the measured biometric data with a smartphone or the like. There are commercially available a large number of such medical measurement apparatuses that have been downsized to a portable level such as, for example, electrocardiographs, sphygmomanometers, heart rate meters, pedometers, and activity meters, which are easily available for the user, allowing the user to acquire and refer his/her biometric data in the course of daily life.

A user who uses such apparatuses may take advantage thereof to reshape his/her daily life so that values of respective types of biometric data fall within appropriate ranges recommended by an expert such as a doctor, in order to keep his/her health in a good condition, or facilitate recovery from a disease. In addition, it is assumed that medical personnel may utilize such biometric data for diagnosis and treatment of disease of the user.

For example, Patent Document 1 discloses that advice information for a patient is automatically generated based on biometric information of the patient received from a terminal belonging to the patient, and transmitted to the terminal belonging to the patient. In addition, Patent Document 2 discloses that biologic information to a first candidate terminal is transmitted, upon determining that an anomaly exists in the body of a subject to be assisted through analysis of the biometric information by an information analysis unit of a home-use communication apparatus.

As has been described above, the advice information is generated as data for the patient in Patent Document 1 whereas, in Patent Document 2, biometric information of the patient is transmitted to a terminal of a family doctor as data directed to the doctor.

### CITATION LIST

### Patent Literature

Patent Document 1: JP 2006-259827 A

Patent Document 2: JP 5917754 B

### SUMMARY OF INVENTION

### Technical Problem

As has been described above, a conventional device either generates advice information for a patient from the patient's biometric information received from a terminal for the patient or, in a case where it has been determined that an anomaly exists in the body of a subject to be assisted such as the patient, transmits the patient's biometric information to another preliminarily set terminal (e.g., a terminal of a doctor).

Therefore, for example, the conventional device neither generates any type of information for a user, who is the patient, based on the user's biometric information, nor generate data for a doctor based on the same biometric information and provide the doctor with the data.

The present invention, which has been made in consideration of the above circumstances, intends to provide a data generation device, a method, and a non-transitory storage medium storing a program configured to generate data corresponding to activities that a user and a medical professional are supposed to perform, respectively, based on the same biometric data.

### Solution to Problem

In order to solve the problems described above, the present disclosure adopts the following configuration.

Specifically, a data generation device according to a first aspect of the present disclosure includes a biometric data acquisition unit configured to acquire biometric data of a user, a generation unit configured to generate, based on the biometric data, user-oriented data directed to the user, and medical professional assistance data directed to a medical professional, the medical professional assistance data assisting an activity performed by the medical professional in relation to the user, an access control unit configured to control knowability about content of the medical professional assistance data, and a data presentation unit configured to present the user-oriented data to the user, wherein the access control unit generates access-enabling data that releases the limitation on the knowability or enhances the knowability, to a specified medical professional, about the content of the medical professional assistance data, and the data presentation unit displays, or transmits an instruction for printing, the access-enabling data together with the user-oriented data.

In the above-described configuration, user-oriented data and medical professional assistance data directed to a medical professional are generated from the same biometric data of the user acquired by the biometric data acquisition unit. The user-oriented data is generated at least in a manner allowing the user to view the content thereof. The medical professional assistance data is assumed to include medical expertise, and include data for assisting the medical professional when the medical professional perform an appropriate activity on the user such as diagnosis or treatment in relation to the user. For example, the medical professional assistance data may include, in addition to detailed information of the biometric data, names or diagnostic information of one or more diseases likely to be contracted, which are inferred from the detailed information. In addition, the medical professional assistance data may include a content, which is generally incomprehensible to the user or a content having too large data volume which is cumbersome to be viewed by the user, although the data itself is not medical expertise. Here, the term “medical professional” includes all the people engaged in medical practice such as doctors and nurses, and may indicate doctors but may also indicate at least those engaged in medical practice without being limited to doctors. Activities performed by the medical professional in relation to a user are assumed to include a wide variety of activities depending on the category of medical professional, without being limited to such as diagnosis or treatment. Here, the term “same biometric data” means that the biometric data that are the basis for generating the user-oriented data and the medical professional assistance data are common, without being limited to a single biometric data, a biometric data acquired by a one-time measurement, or one type of biometric data.

In the data generation device, there is no limitation on the timings at which the user-oriented data and the medical professional assistance data are respectively generated. The user-oriented data and the medical professional assistance data may be generated simultaneously with acquisition of the biometric data, or may be generated at an arbitrary timing after acquisition of the biometric data (e.g., during medical consultation of the user, etc.). In addition, the timing at which the user-oriented data is generated and the timing at which the medical professional assistance data is generated may be identical or different.

A medical professional or a medical institution may lend the user a data generation device or a device that forms a part thereof, and the medical professional may acquire medical professional assistance data from the data generation device or part thereof from which the user has acquired biometric data of the user by a predetermined amount, or for a predetermined time period. On this occasion, a part of the processing by the data generation device may be performed by the server. With such an aspect of usage, in a case where the data generation device is constituted by a sphygmomanometer and a smartphone, for example, the medical professional or medical institution may lend the user only the sphygmomanometer, or the medical professional or medical institution may lend the user the sphygmomanometer and the smartphone.

Biometric data is data related to the user's body, such as electrocardiographic data, blood pressure data, heart rate data. The user-oriented data is data supposed to be provided to the user with regard to acquisition of biometric data, activities performed by the medical professional in relation to the user, or activities the user is recommended to perform. The user-oriented data includes, for example, data that recommends the user to receive user-related activities from the medical professional, in a case where the biometric data indicates an anomaly. In addition, without being limited thereto, the user-oriented data also includes data that, in a case where the user is scheduled to receive an activity related to the user from the medical professional, guides the user to receive the scheduled activity. In addition, the user-oriented data also includes data that, in a case where biometric data by a predetermined amount or for a predetermined time period is acquired, recommends the user to receive the user-related activity from the medical professional. Therefore, generation of biometric data is not limited to when the generated data indicates an anomaly.

In addition, the above-described configuration presents the user-oriented data to the user via the data presentation unit.

In the above-described configuration, the data generation device includes the access control unit configured to control the knowability about the content of the medical professional assistance data. The medical professional assistance data generated from the biometric data of the user is assumed to include, as has been described above, detailed information of the biometric data, medical expertise such as names or diagnostic information of diseases likely to be contracted including the name or diagnostic information of the disease that is most likely to be contracted from the detailed information. Such medical professional assistance data is a type of data whose content may not be accurately comprehensible to the user viewing the data without advice, guidance, or explanation by an expert such as the medical professional, or data which may not be necessarily required to be known to the user. In addition, such medical professional assistance data may also include information related to privacy of the user as an individual. From the viewpoint of such user privacy, the medical professional assistance data must be rendered so that not only a third party other than a medical professional but also a medical professional other than a specified medical professional involved in an activity related to the user cannot easily know the content thereof. In addition, the extent or range to which the medical professional assistance data generated from the biometric data of a user should be disclosed to the user may depend on the nation (or society), or further on the system, and therefore it is necessary to appropriately control the user's knowability about the medical professional assistance data generated from the user's own biometric data, in accordance with the nation (or society), or the system. Therefore, in the above-described configuration, the access control unit controls the knowability about the content of the medical professional assistance data. Knowing the content of the medical professional assistance data means that it is not sufficient to simply access the medical professional assistance data represented in a variety of forms or aspects, but requires comprehension of the content of the medical professional assistance data. In addition, the form or aspect of representing the medical professional assistance data here does not only mean representation, such as language or image, on the premise of recognition by a human, but also includes compression, segmentation, encryption and the like. On this occasion, for example, at a stage of accessing medical professional assistance data as physical data, or knowing an address or the like of an area where medical professional assistance data is stored and only instructing the terminal to access the area, the content of the medical professional data is in a state that cannot be read by a device or a human, in other words, has not yet reached a state that any medical professional can comprehend the content of the medical professional assistance data, in a case where the medical professional assistance data has been subjected to encryption or the like, or additional authentication or the like is required to access into an area having the medical professional assistance data stored therein, and therefore it can be said that the medical professional has become able to comprehend, i.e., know the content of the medical professional assistance data only after the medical professional has brought the medical professional assistance data, which is subjected to decryption or the like and converted into plain text, being in a state that can be displayed on a display unit of a medical professional terminal, or printed by a printing device, or after the medical professional has established authentication or the like using a password or the like to access into the area having the medical professional assistance data stored therein, and has brought the medical professional assistance data stored in the area being in a state that can be displayed on the display unit of the medical professional terminal, or printed by the printing device. In addition, controlling the knowability about the content of the medical professional assistance data includes limiting the knowability about the content of the medical professional assistance data to a specified or unspecified target personnel, and also releasing the limitation thereon for the specified target personnel. In addition, the controlling also includes enhancing the knowability about the content of the medical professional assistance data by the specified target personnel, making it easier to know the content. However, the knowability cannot be said to be limited in a case where anyone lacking expertise related to the medical professional assistance data cannot, but anyone having such expertise can, comprehend the content thereof.

Such access control includes, for example, encrypting the medical professional assistance data with an arbitrary encryption method. On this occasion, a target medical professional preliminarily possesses an encryption key of the encryption method that allows for decrypting the encrypted medical professional assistance data and referring to the data. Here, encrypting the medical professional assistance data prevents any person without possessing the encryption key of the encryption method from decrypting the medical professional assistance data, and therefore the person is provided with limited knowability about the content. In contrast, a medical professional possessing the encryption key of the encryption method thus can decrypt the medical professional assistance data and comprehend the content thereof, and therefore the limitation on the knowability about the medical professional assistance data has been released for the target medical professional. In addition, the encryption key may be transmitted to a medical professional when the medical professional is attempting decryption. In addition, access control can also be performed in a manner similar to encryption by encoding the medical professional assistance data according to some uncommon encoding scheme. The encoding scheme is already known to a terminal or the like used by a target medical professional, and decoding with a decoding scheme corresponding to the encoding scheme allows the medical professional to comprehend the content of the corresponding medical professional assistance data. In addition, the data generation device that initially generates medical professional assistance data is under administration by the user. Therefore, access control of the medical professional assistance data may also be realized in a manner such as prohibiting the generated medical professional assistance data to be displayed on the data generation device, transmitted from the data generation device, or output from the data generation device, for those other than a specific medical professional. For example, in a case where the data generation device includes a sphygmomanometer including a data measurement unit and a smartphone, it is also conceivable to prohibit the generated medical professional assistance data to be displayed on a display unit of the sphygmomanometer or the smartphone, transmitted from the sphygmomanometer or the smart phone, or output from the sphygmomanometer or the smart phone.

Therefore, with the data generation device according to the first aspect of the present disclosure, there is an extremely low possibility that a third party other than the target medical professional can acquire knowledge of the content, and therefore personal information including information related to the user's personal biometric data is protected.

Furthermore, it suffices that the data generation device according to the first aspect of the present disclosure may be a device that can acquire biometric data of a user to generate user-oriented data and medical professional assistance data for a medical professional, and execute a program for performing access control of the medical professional assistance data, and the device may be, for example, a wearable apparatus (e.g., a smart phone, or a wristwatch-type wearable terminal), an activity meter, or a stationary device (e.g., a personal computer). Furthermore, it suffices that the data generation device can acquire biometric data, regardless of whether it is worn by the user. For example, a detection device that can detect electrocardiographic signals may be attached to the user, and the data generation device may acquire electrocardiographic data (a piece of biometric data) from the detection device and execute the program described above.

The data generation device may also be configured as a single device, or configured to include a plurality of devices operating in conjunction with each other. For example, the data generation device may be configured to include a sphygmomanometer and a smartphone serving a terminal that can communicate with the sphygmomanometer.

In addition, the data presentation unit included in the data generation device is not limited to a single unit, and a plurality of data presentation units may be provided therein. In a case where the data generation device is configured to include a sphygmomanometer and a smartphone, as with the example described above, the sphygmomanometer and the smart phone respectively include a data presentation unit, so that the data generation device turns out to include two data presentation units. Also the generation unit, the access control unit, and the biometric data acquisition unit included in the data generation device are not respectively limited to a single unit, and a plurality of generation units or the like may operate in conjunction with each other, or in a manner sharing functions, depending on the configuration of the data generation device. In addition, a part of the processing in each unit of the data generation device may be performed by a server.

In a data generation device according to a second aspect of the present disclosure, the access control unit generates access-enabling data that releases the limitation on the knowability or enhances the knowability, to a specified medical professional, about the content of the medical professional assistance data.

The above-described configuration generates access-enabling data for releasing the limitation on the knowability or enhancing the knowability about the content of the medical professional assistance data to the specified medical professional. Here, the access-enabling data for releasing the limitation on knowability is a type of data for releasing the limitation applied on the knowability of the medical professional assistance data, such as a password in a case where the password has been set to open a file. In addition, the access-enabling data for enhancing the knowability is not a type of data that releases the limitation by itself, such as an address indicating the area having the medical professional assistance data stored therein, but is a type of data that facilitates acquisition of knowledge of the content of medical professional assistance data with acquisition of that data, while substantially limiting access to by anyone who does not know the data. The idea is to incorporate, in the access-enabling data, a type of data that enhances the knowability of a specified medical professional including a case where substantial limitation is yet to be fulfilled, or a case where the data itself cannot accomplish control but access control is performed by a combination with another access control tools (e.g., ID and password for logging in a system used by a medical professional). Any medical professional having acquired such access-enabling data can know the content of the medical professional assistance data. Such access-enabling data may be, for example, in a case where the medical professional assistance data is encrypted by an arbitrary encryption method, an encryption key for the encryption method. The access control unit may generate an encryption key and transmit the encryption key to a terminal used by a specified medical professional when the specified medical professional attempts decryption. In addition, it is also possible to display a one-time password that is an access-enabling data on the data presentation unit of the user's data generation device (e.g., a touch panel display of a smartphone), and the one-time password is then visually recognized by a specified medical professional or read aloud by the user, whereby the medical professional can know the one-time password and input it to the terminal that he or she uses. Using the access-enabling data generated by the access control unit in the aforementioned manner, the limitation on the knowability of the content of the medical professional assistance data is released or relaxed for a specified medical professional, and therefore it is possible to prevent anyone other than the specified medical professional from knowing the medical professional assistance data and keep the data secret. Even when encoded according to a certain uncommon encoding scheme, an effect close to encryption is expected. The encoding scheme described above is known in advance to a terminal or the like used by a medical professional, and the medical professional may refer corresponding medical professional assistance data by decoding the data using a decoding scheme corresponding to the encoding scheme.

The access-enabling data may be expressed in a two-dimensional code. In addition, it may be set such that the two-dimensional code may be encrypted so that only meaningless content is displayed even when the two-dimensional code is read, and that the medical professional assistance data can be accessed only after inputting a password or the like possessed by only a medical professional.

In addition, the access-enabling data may be encrypted. Furthermore, encoding the access-enabling data according to a certain uncommon encoding scheme is also expected to exhibit an effect close to encryption. The encoding scheme is already known to a terminal or the like used by a medical professional, and therefore the medical professional can know the content of the corresponding medical professional assistance data by acquiring the access-enabling data by decoding the data using a decoding scheme corresponding to the encoding scheme.

In addition, the access-enabling data may be an address of a server having the medical professional assistance data stored therein.

In a data generation device according to a third aspect of the present disclosure, the access-enabling data is a password to be presented to the specified medical professional to release the limitation on the knowability, to the specified medical professional, about the content of the medical professional assistance data.

In the above-described configuration, presenting, to the specified medical professional, a password generated by the access control unit to release the limitation on the knowability to the specified medical professional about the content of the medical professional assistance data allows the specified medical professional to know the content of medical professional assistance data using the password.

In a data generation device according to a fourth aspect of the present disclosure, the data presentation unit displays, or transmits an instruction for printing, the access-enabling data together with the user-oriented data.

In the above-described configuration, displaying, or transmitting an instruction for printing to the printing device internal or external to the device, the access-enabling data together with the user-oriented data, whereby the user can visit a hospital or the like to receive an activity by a medical professional in accordance with the user-oriented data, and present the access-enabling data to the medical professional by handing the access-enabling data to the medical professional as a printed material printed by the printing device having received the print instruction, or by displaying the data on a display or the like. Using the access-enabling data on the spot, a medical professional can access the medical professional assistance data which can be known by only the medical professional, and improve the activity performed on the user, referring to the medical professional assistance data. Here, the phrase “together with” is not limited to a case where the user-oriented data and the access-enabling data are displayed on a same screen or printed on a same paper sheet, but also includes a case where the data spreads across two or more pages, intending that correspondence relation between the user-oriented data and the access-enabling data is displayed or printed in an explicit manner.

Therefore, the data generation device according to the fourth aspect of the present disclosure allows the user to present access-enabling data (and also user-oriented data) to a medical professional when receiving an activity related to the user from the medical professional, and the access-enabling data allows for easily providing medical professional assistance data to the medical professional without intervention of a network or the like.

A data generation device according to a fifth aspect of the present disclosure further includes a transmission unit configured to transmit the medical professional assistance data to a server, and the access control unit sets an area in the server, where the medical professional assistance data is stored, to have a limitation on the knowability about the content of the medical professional assistance data stored in the area.

In the above-described configuration, transmitting the medical professional assistance data to the server makes it unnecessary to store the data in the data generation device, and therefore even when volume of the medical professional assistance data is large, the storage capacity of the data generation device can be suppressed, allowing downsizing and reduced price of the data generation device. With the limited knowability about the content of the medical professional assistance data stored in the server, it is possible to manage data unitarily and securely while preserving confidentiality of the data. Additionally, in the event that the terminal of the medical professional fails or is lost or the like, the medical professional can acquire the medical professional assistance data using a terminal that can access the server.

In a data generation device according to a sixth aspect of the present disclosure, the access control unit generates an address of a server having the medical professional assistance data stored therein, as access-enabling data for releasing the limitation on the knowability or enhancing the knowability, to the specified medical professional, about the content of the medical professional assistance data, and the data presentation unit displays, or transmits an instruction for printing, the address together with the user-oriented data.

In the above-described configuration, the address of the server having data stored therein is generated. The user causing the address to be displayed on the data presentation unit, or printed by a printing device internal or external to the device allows the user to read the address, and a medical professional reading the address via the user allows the medical professional to view and know the content of the medical professional assistance data. More specifically, for example, the user shows the medical professional the address displayed on the data presentation unit of the data generation device or printed on a paper sheet. The medical professional can view and know the content of the medical professional assistance data displayed on the display unit of the terminal by inputting the address to the terminal used by the medical professional. In addition, reading the address displayed on the data presentation unit or printed in the form of a plaintext or a QR code (trade name) with a reading unit such as a camera or a code reader provided on a terminal used by a medical professional allows the medical professional to view and know the content of the medical professional assistance data. In addition, presenting the address to the user also serves as a reminder to the user to ask the medical professional to view the medical professional assistance data.

Here, it is the user possessing the data generation device that first receives the displayed or printed address and therefore the user, for example, can also view the address using any access control tools, but it is impossible for the user (and any third party) to access the medical professional assistance data or decrypt the encrypted data using only the address, because the address in the form of the access-enabling data is realized by a combination of a system, software, a login ID, and the like possessed by only a medical professional, using an arbitrary access control tool, for example. Accordingly, there is no problem with the data generation device presenting the address in a user-recognizable aspect. In addition, the data generation device may present not only the address but also the password for entering the server.

In a data generation device according to a seventh aspect of the present disclosure, the access control unit includes an access destination-specifying unit configured to specify an access destination, having the medical professional assistance data stored therein, as access-enabling data for enhancing the knowability.

In the above-described configuration, the access control unit specifies an access destination having the medical professional assistance data stored therein. By reading the access destination having the medical professional assistance data stored therein, a specified medical professional can enhance the knowability of the content. Here, the access destination of the medical professional assistance data includes data specifying the area having the medical professional assistance data stored therein, and names of files, folders, or directories including the medical professional assistance data. The foregoing items may be specified by data such as user name and ID, time of measurement of biometric data. In addition, the access destination may include a procedure or method for accessing.

In a data generation device according to an eighth aspect of the present disclosure, the access control unit generates limited medical professional assistance data having limited knowability about the content of the medical professional assistance data.

In the above-described configuration, the limited medical professional assistance data is generated as medical professional assistance data having limited knowability about the content of the medical professional assistance data. Since the knowability about the content of the medical professional assistance data is limited, it turns out that only a specified medical professional whose knowability is not limited, or who can release the limitation on the knowability can know the content of the limited medical professional assistance data. In other words, any medical professional (and a third party) other than the specified medical professional is limited from knowing the content of the medical professional assistance data. Therefore, a more flexible access control can be performed in accordance with the target person, such as in a case where it is desired to show the medical professional assistance data to a specified medical professional. The medical professional assistance data is encrypted by an arbitrary encryption method, and a specified medical professional preliminarily possesses an encryption key of the encryption method for decrypting the limited medical professional assistance data to know the content of the medical professional assistance data. The encryption key may have incorporated therein an ID and the like of a medical professional or identification information and the like specific to the terminal used by the medical professional. In addition, an encryption key may be transmitted to the specified medical professional when the medical professional is attempting decryption. In addition, the medical professional assistance data is expected to exhibit an effect close to encryption even when encoded according to a certain uncommon encoding scheme. The encoding scheme is preliminarily known to a terminal or the like used by a specified medical professional, and therefore the specified medical professional can know the content of the corresponding medical professional assistance data by decoding the data using a decoding scheme corresponding to the encoding scheme.

Therefore, the data generation device according to the eighth aspect of the present disclosure allows for including, in the medical professional assistance data, medical data which need not be comprehended by a user who is not a medical professional, or which is expertise for a medical professional. In addition, according to the data generation device according to the present aspect, the limited medical professional assistance data has been encrypted whereby it is very unlikely that a third party other than a specified medical professional can know the content thereof, and therefore personal information including information related to the user's personal biometric data is protected.

In a data generation device according to a ninth aspect of the present disclosure, the generation unit generates the medical professional assistance data as limited medical professional assistance data in a form of data described in a two-dimensional code.

In the above-described configuration, the limited medical professional assistance data is described in the form of a two-dimensional code, and therefore the content thereof cannot be comprehended at a glance. The content of the two-dimensional code is set so that it cannot be accessed without using software possessed by only a specified medical professional. The two-dimensional code, for example a QR code, can include information in vertical and horizontal directions (two dimensions), in which a larger amount of data can be stored than a bar code. It suffices that the two-dimensional code is associated with the specific medical professional assistance data. The two-dimensional code may be the address of the server having the medical professional assistance data stored therein. In addition, it may be set such that the two-dimensional code is encrypted so that only meaningless content is displayed even when the two-dimensional code is read, and that the medical professional assistance data can be accessed only after inputting a password or the like possessed by only a medical professional.

According to a tenth aspect of the present disclosure, the data presentation unit displays, or transmits, to the outside of the apparatus, an instruction for printing, the medical professional assistance data together with the user-oriented data.

In the above-described configuration, displaying the medical professional assistance data together with the user-oriented data or printing them by the printing device internal or external to the device, whereby the user can visit a hospital or the like to receive an activity by a medical professional in accordance with the user-oriented data, and present the medical professional assistance data to the medical professional by handing the medical professional assistance data to the medical professional as a printed material, or by displaying the data on a display or the like. A medical professional can know the content thereof by viewing the medical professional assistance data on the spot, and improve activities for the user, referring to the medical professional assistance data.

Therefore, the data generation device according to the tenth aspect of the present disclosure allows the user to present medical professional assistance data (and also user-oriented data) to a medical professional when receiving an activity related to the user from the medical professional, and the medical professional assistance data allows for easily providing medical professional assistance data to the medical professional without intervention of a network or the like.

A data generation device according to an eleventh aspect of the present disclosure further includes a display unit configured to display an interface that enables the user to execute a process of transmitting at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

In the above-described configuration, a display unit of the data generation device further provided, the display unit displays an interface for starting a process of transmitting at least one of medical professional assistance data, limited medical professional assistance data having limited knowability about the content of the medical professional assistance data, and access-enabling data for releasing the limitation on the knowability or enhancing the knowability about the content of the medical professional assistance data to a specified medical professional, and the user can use the interface to transmit, from the data generation device, at least one of the medical professional assistance data, limited medical professional assistance data having limited knowability about the content of the medical professional assistance data, and access-enabling data for releasing the limitation on the knowability or enhancing the knowability about the content of the medical professional assistance data to a specified medical professional. The interface may be of any type, for example, a button of a Graphic User Interface (GUI), as long as the user can input an instruction for data transmission, and a user pressing the button of the GUI causes the data generation device to transmit at least one of the medical professional assistance data, limited medical professional assistance data having limited knowability about the content of the medical professional assistance data, and access-enabling data for releasing the limitation on the knowability or enhancing the knowability about the content of the medical professional assistance data to a specified medical professional.

A data generation device according to a twelfth aspect of the present disclosure is configured to store, in a portable storage medium at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, and the data presentation unit presents user-oriented data indicating that at least one of the data is stored in the storage medium, and prompting to pass the storage medium to the medical professional.

In the above-described configuration, at least one of medical professional assistance data, limited medical professional assistance data having limited knowability about the content of the medical professional assistance data, and access-enabling data for releasing the limitation on the knowability or enhancing the knowability about the content of the medical professional assistance data to a specified medical professional is stored in a portable storage medium, and the data presentation unit has presented thereon user-oriented data indicating that at least one piece of the data is stored in the portable storage medium, and prompting to pass the storage medium to the medical professional. Therefore, a user can provide the medical professional with at least one of medical professional assistance data, limited medical professional assistance data having limited knowability about the content of the medical professional assistance data, and access-enabling data for releasing the limitation on the knowability or enhancing the knowability about the content of the medical professional assistance data to a specified medical professional, by passing the storage medium to the medical professional when receiving an activity related to the user from the medical professional. The portable storage medium may be, for example, a memory card, or a Universal Serial Bus (USB) memory, without being limited thereto.

A data generation device according to a thirteenth aspect of the present disclosure further includes a setting unit configured to set at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data to be transmittable to the terminal of the medical professional, and causing the data presentation unit to present user-oriented data that guides the user to transmit, to the terminal of the medical professional, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, so that the medical professional can know the medical professional assistance data when the user receives an activity related to the user from the medical professional.

In the above-described configuration, when the user receives an activity related to the user from the medical professional, in order to allow the medical professional to know the medical professional assistance data, the user-oriented data is presented on the data presentation unit, which guides the user to transmit, to the terminal of the medical professional, at least one of the medical professional assistance data, limited medical professional assistance data having limited knowability about the content of the medical professional assistance data, and access-enabling data for releasing the limitation on the knowability or enhancing the knowability about the content of the medical professional assistance data to a specified medical professional, and at least one of the medical professional assistance data, limited medical professional assistance data having limited knowability about the content of the medical professional assistance data, and access-enabling data for releasing the limitation on the knowability or enhancing the knowability about the content of the medical professional assistance data to a specified medical professional can be set transmittable to the terminal of the medical professional, and therefore the user can perform transmission when, for example, the user receives an activity related to the user from the medical professional, and make the medical professional assistance data available to a specified medical professional currently performing medical examination, and the medical professional, when performing a user-related activity on the user, can receive, with the terminal of the medical professional, at least one of the medical professional assistance data, limited medical professional assistance data having limited knowability about the content of the medical professional assistance data, and access-enabling data for releasing the limitation on the knowability or enhancing the knowability about the content of the medical professional assistance data to a specified medical professional, and immediately know, with the terminal, the content of the medical professional assistance data related to the user currently in medical consultation. In addition, the timing at which the user performs transmission is not limited to the foregoing, when the user receives an activity related to the user from a medical professional, in order to allow the medical professional to know the medical professional assistance data, a guidance may be provided to transmit at a timing such as, for example, medical interview prior to medical examination and the like by the medical professional after the user arrived at the medical institution, or to transmit at the timing of establishing the reservation for the reservation application made by the user to receive an activity related to the user from a specified medical professional.

A data generation device according to a fourteenth aspect of the present disclosure further includes a transmission unit configured to transmit, according to the near-field wireless communication method, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, and the access control unit, upon authenticating that the transmission destination is the terminal of the medical professional, transmits, to the terminal of the medical professional, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

In the above-described configuration, the data generation device can transmit, according to the near-field wireless communication method, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, and the access control unit, upon authenticating that the transmission destination is the terminal of the medical professional, can transmit at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data. Here, the near-field wireless communication method may be any communication method without being limited to any particular method.

A fifteenth aspect of the present disclosure includes a display unit configured to display an interface that accepts permission of the user for association with the terminal of the medical professional, and the access control unit, upon accepting a request from the terminal of the medical professional and authenticating that the request is from the terminal of the medical professional associated based on the permission, transmits, to the terminal of the medical professional associated based on the permission, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

In the above-described configuration, the data generation device and the terminal of the medical professional are preliminarily associated with each other, based on permission of the user accepted via the interface displayed on the display unit. Subsequently, the access control unit, upon accepting a request from a terminal of a medical professional and authenticating that the request is from a preliminarily associated terminal of a medical professional, transmits, to the preliminarily associated terminal of the medical professional, at least one of medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data. The user can select whether or not to permit association with the terminal of the medical professional, via the interface displayed on the display unit. Subsequently, in a case where the user has permitted association, the data is transmitted to the terminal of the medical professional with which the data is associated, without the need of respective permissions or operations by the user upon the access control unit accepting a request to transmit the data to the terminal of the medical professional, when the user receives an activity related to the user from the medical professional.

A data generation device according to a sixteenth aspect of the present disclosure presents, on the data presentation unit, user-oriented data that guides to display, or transmit an instruction for printing, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, when the user receives, from a medical professional, an activity performed by the medical professional in relation to the user, and the data presentation unit displays, or transmits an instruction for printing, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

In the above-described configuration, when the user receives, from a medical professional, an activity performed by the medical professional in relation to the user, the data presentation unit presents user-oriented data including a guidance to display, or transmit an instruction for printing, at least one of medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, and the data generation device displays, or transmits an instruction for printing, at least one of medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, and therefore it becomes easy for the medical professional to access at least one of medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, by referring to the display or print when the user receives, from the medical professional, an activity performed by the medical professional in relation to the user. In addition, the access-enabling data may be any information for accessing medical professional assistance data (information for specifying access destination) such as, for example, file name and password of medical professional assistance data, residence address and password of medical professional assistance data.

A data generation device according to a seventeenth aspect of the present disclosure includes a display unit configured to display an interface that accepts permission of the user for association with the terminal of the medical professional and, the access control unit, upon accepting a request from the terminal of the medical professional and authenticating that the request is from the terminal of the medical professional associated based on the permission, causes the data presentation unit to display, or transmits an instruction for printing, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

In the above-described configuration, the data generation device and the terminal of the medical professional are preliminarily associated, based on the user permission accepted via the interface displayed on the display unit. Subsequently, the access control unit, upon accepting a request from a terminal of a medical professional and authenticating that the request is from a preliminarily associated terminal of a medical professional associated based on the permission, transmits an instruction that causes the data presentation unit to display or print at least one of medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data. The user can select whether or not to permit association with the terminal of the medical professional, via the interface displayed on the display unit. Subsequently, in a case where the user has permitted association, it is possible to cause the data presentation unit to display, or transmit an instruction for printing, the data, without the need of respective permissions or operations by the user, in a case where the user has accepted the request of displaying or printing the data when receiving an activity related to the user from the medical professional.

A data generation device according to an eighteenth aspect of the present disclosure further includes a determination unit configured to determine whether or not the biometric data indicates an anomaly, and in a case where it has been determined that the biometric data indicates an anomaly, the user-oriented data includes data that recommends the user to receive the activity from the medical professional.

Specifically, the data generation device, upon determining that symptoms such as uneven heart beat are occurring based on determination of the biometric data, for example, generates user-oriented data that recommends the user to receive medical practice by a medical professional such as a cardiology doctor, for example, who professionally treats symptoms including uneven heartbeat, and generates, for the medical professional, medical professional assistance data presenting the user's detailed electrocardiographic data and suggesting that the electrocardiographic data indicates that the user is probably suffering from arrhythmia.

As has been described above, the data generation device according to the eighteenth aspect of the present disclosure allows for recommending the user to receive an activity such as medical examination by the medical professional and generating detailed information of the biometric data acquired from the same biometric data and name or diagnostic information of the disease that is likely to be associated with the biometric data. Therefore, according to the present configuration, it becomes possible to generate, based on the biometric data indicating an anomaly, user-oriented data that recommends the user to receive to an activity by a medical professional such as medical examination, and also generate medical professional assistance data for passing appropriate medical information such as biometric data to the medical professional.

In a data generation device according to a nineteenth aspect of the present disclosure, the generation unit generates, by incorporating into the medical professional assistance data, at least one of biometric data of the user acquired from after the determination by the determination unit to when the user receives the activity from the medical professional, medical data related to the user, and lifestyle data.

In the above-described configuration, when the user receives an activity related to the user such as medical examination by the medical professional, at least one of biometric data of the user for a time period from determination that the biometric data indicates anomaly to reception of an activity such as medical examination by the medical professional, medical data related to the user, and lifestyle data is incorporated in the medical professional assistance data, and therefore the medical professional can refer to the progress of the biometric data or the like since the determination of anomaly. As a result, the medical professional can accurately grasp the biometric state and/or medical condition of the user according to the progress data of the biometric data or the like originated from determination of anomaly. In addition, medical data is a type of data related to medical care, and the lifestyle data is a type of data that may affect the biometric data related to the lifestyle of the user.

In a data generation device according to a twentieth aspect of the present disclosure, the generation unit generates, by incorporating into the medical professional assistance data, in accordance with the determination by the determination unit, at least one of biometric data of the user prior to the determination, medical data related to the user, and lifestyle data, the three sets of data having been preliminarily set.

In the above-described configuration, at least one of biometric data of the user prior to the determination that the biometric data indicates anomaly, medical data related to the user, and lifestyle data is incorporated in the medical professional assistance data, when the user receives an activity related to the user such as medical examination by the medical professional, and therefore the medical professional can refer to progress of the biometric data prior to the determination of anomaly. As a result, medical professional can accurately grasp the biometric condition and/or medical condition of the user according to the progress data of the biometric data, which may lead to determination of anomaly. In addition, the medical data is a type of data related to medical care, whereas the lifestyle data is a type of data that may affect the biometric data related to the living of the user. In addition, the range and the content of the biometric data or the like to be incorporated in the medical professional assistance data is preliminarily set in accordance with the result of determination by the determination unit. In the foregoing manner, it is possible to provide useful data to the medical professional in accordance with the result of determination by the determination unit.

In a data generation device according to a twenty-first aspect of the present disclosure, the data presentation unit further presents to prompt the user to inform the medical professional of existence of medical professional assistance data, or pass the medical professional assistance data to the medical professional.

In the above-described configuration, the data presentation unit displays or prints medical professional assistance data, and also presents to prompt the user to inform the medical professional of existence of the access-enabling data or pass it to the medical professional, and therefore it becomes possible, even in a case where the user has forgotten to present the medical professional assistance data to the medical professional, that the presentation reminds the user to present the medical professional assistance data to the medical professional allowing the medical professional to refer the content thereof.

Therefore, with the data generation device according to the twenty-first aspect of the present disclosure, the medical professional can refer to the content of the medical professional assistance data more reliably than the seventeenth aspect only. Additionally, in a case where the biometric data of the user indicates that a plurality of events are occurring, the medical professional can refer to medical professional assistance data respectively corresponding thereto.

In a data generation device according to a twenty-second aspect of the present disclosure, the determination unit determines a level indicating the degree of the anomaly based on the biometric data, and determines that the biometric data is indicating an anomaly in a case where the level is equal to or higher than a threshold value.

In the above-described configuration, the determination unit determines the level indicating the degree of the anomaly based on the biometric data. The determination unit, having preliminarily set a range of level for each type based on the physical quantity of the biometric data, determines the level in accordance with a value of a physical quantity included in the biometric data, and determines that the biometric data indicates an anomaly when the level of some biometric data is equal to or higher than a threshold value. As has been described above, fine-setting a threshold value indicating the anomaly level in the biometric data allows the data generation device to determine for each biometric data whether or not an anomaly exists. Additionally, in a case of being determined for a certain level or more (equal to or higher than a threshold level), the data generation device may have the biometric data recorded or stored therein, and present it to the medical professional when the user receives an activity related to the user such as medical examination from the medical professional.

In a data generation device according to a twenty-third aspect of the present disclosure, the medical professional assistance data includes anomaly-specifying data for specifying data related to an anomaly in the acquired biometric data.

In the above-described configuration, medical professional assistance data includes anomaly-specifying data for specifying anomaly-related data in the biometric data, and therefore the anomaly-specifying data allows for easily grasping the part of the biometric data to be checked. It is possible to provide the medical professional with useful data to know the state of the user.

In addition, the data generation device according to the present disclosure can be configured as a data generation method executed by a computer or a program for causing a computer to function as each unit of the data generation device.

In a data generation device according to yet another aspect, the two-dimensional code is acquired by an image acquisition unit connected to the terminal of the medical professional.

In the above-described configuration, the two-dimensional code presented by the data generation device is acquired by the image acquisition unit of the medical professional, and therefore it becomes possible for the medical professional to immediately decode the two-dimensional code to refer to the access-enabling data or the medical professional assistance data. The image acquisition unit may be connected to the terminal for the medical professional, or may be built in the terminal for the medical professional.

### Advantageous Effects of Invention

According to the present invention, it is possible in one aspect to provide a data generation device, a method, and a non-transitory storage medium storing a program configured to generate data corresponding to activities that a user and a doctor are supposed to respectively perform based on the same biometric data, in a case where the biometric data indicates an anomaly of the user.

### BRIEF DESCRIPTION OF DRAWINGS

FIG. 1 illustrates an overview of a system including a data generation device, a wristwatch-type wearable terminal, and a medical professional terminal, according to a first embodiment;

FIG. 2 schematically illustrates an example of a hardware configuration of the data generation device according to the first embodiment;

FIG. 3 illustrates an example of a part of a software configuration of the data generation device according to the first embodiment;

FIG. 4 is a flow chart schematically illustrating an example of a processing procedure related to a data generation device and a dedicated terminal for a medical professional according to the first embodiment;

FIG. 5 illustrates an overview of a system including a data generation device, a wristwatch-type wearable terminal, a medical professional terminal, and a server connected via a network according to a second embodiment;

FIG. 6 schematically illustrates an example of a hardware configuration of the data generation device according to the second embodiment;

FIG. 7 illustrates an example of a part of the software configuration of the data generation device according to the second embodiment;

FIG. 8 is a flowchart schematically illustrating an example of a processing procedure related to the data generation device and the medical professional dedicated terminal according to the second embodiment;

FIG. 9 illustrates an example of a part of the software configuration of a data generation device according to a third embodiment; and

FIG. 10 illustrates an example of a part of the software configuration of a data generation device according to a fourth embodiment.

### DESCRIPTION OF EMBODIMENTS

An embodiment according to an aspect of the present invention (hereinafter, also referred to as “the present embodiment”) will be described below with reference to the drawings. Note that, in the following embodiment, parts denoted by the same reference numbers perform the same operations, and a repetitive explanation will be omitted.

### First Embodiment

### Overview

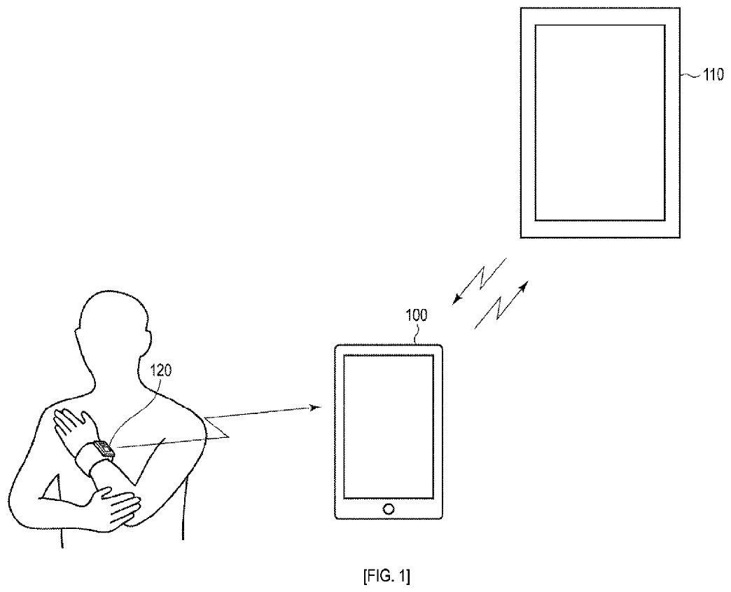

First, an overview of a data generation device of the present invention will be described, referring to FIG. 1.

FIG. 1 schematically illustrates an example of a data generation device 100, a medical professional terminal 110, and a biometric data measurement device 120 according to the example described in the overview.

The data generation device 100 receives, via near-field wireless communication, biometric data acquired by the biometric data measurement device 120 from a user's body, and determines whether or not the biometric data indicates an anomaly. Subsequently, in a case where the data generation device 100 has determined that an anomaly exists, the data generation device 100 generates user-oriented data that recommends the user to receive medical examination (also referred to as receive medical consultation) by a medical professional. Furthermore, the data generation device 100 generates, from the biometric data determined to be indicating an anomaly, medical professional assistance data including data assumed to be useful for medical examination and medical practice by a medical professional. The medical professional assistance data may be set as limited medical professional assistance data which is encrypted and whose content cannot be read without being decrypted using a cryptographic key or the like. In addition, the limited medical professional assistance data may also be set to be encoded by a certain algorithm and decoded by a corresponding algorithm. For example, the limited medical professional assistance data may be encoded in a two-dimensional code, and also the two-dimensional code may be encrypted. In the following, it is intended that the medical professional assistance data indicates data whose content can be known by anyone having acquired the medical professional assistance data, and the limited medical professional assistance data, which is the medical professional assistance data being confidential with limited knowability about the content thereof, indicates data whose content cannot be known without a cryptographic key or a decryption device.

The biometric data measurement device 120, by being worn (or held) by the user, measures biometric data of the user and transmits the biometric data to the data generation device 100. The biometric data may be any data related to a living body, for example, electrocardiographic data, blood pressure data, or heart rate data.

The medical professional terminal 110 receives the medical professional assistance data generated by the data generation device 100, and the medical professional can view the content of medical professional assistance data via the medical professional terminal 110. Furthermore, the medical professional terminal 110 may be set to acquire, by a built-in or connected image acquisition unit, a two-dimensional code displayed on the data generation device 100 or printed on a printed out paper or the like, read the two-dimensional code with dedicated software for a medical professional, thereby allowing the medical professional to read the content of the two-dimensional code (i.e., medical professional assistance data). In addition, the two-dimensional code generated by encoding the medical professional assistance data may itself be encrypted, and in such a case, the medical professional is allowed to read the content of the two-dimensional code (corresponding to the limited medical professional assistance data) by the medical professional inputting a known password (corresponding to the access-enabling data).

As has been described above, the data generation device of the present embodiment acquires the biometric data detected by the biometric data measurement device 120 and, in a case where the biometric data is determined and determined to be indicating an anomaly, generates user-oriented data for the user that recommends the user to receive medical examination by a medical professional, and generates medical professional assistance data for the medical professional for assisting the medical professional with medical examination and treatment of the user, the medical professional assistance data including medical expertise, which is acquired from the biometric data, and/or details of biometric data. Therefore, the data generation device of the present embodiment can recommend the user to receive medical examination by a medical professional, triggered by the biometric data indicating an anomaly, and provide detailed medical data with regard to the biometric data indicating an anomaly of the user to the medical professional performing medical examination of the user based on the same biometric data, and therefore it becomes more likely that the user can receive appropriate medical examination and treatment from the doctor. Furthermore, the data generation device allows the medical professional to read real biometric data indicating occurrence of an anomaly, and therefore facilitates selection for an optimal inspection during medical examination, and further increases the possibility of omitting or reducing the inspection for the selection, thereby making it possible to quickly perform required and optimal inspection, diagnosis, and treatment. In a case where the biometric data indicates an anomaly due to a disease requiring emergency treatment, it often happens that reduction of time taken until treatment and/or time taken from medical examination to treatment has a positive effect on progress and recovery of the user's disease, and therefore a preferable effect on progress and recovery of the user's disease is expected for the data generation device.

### CONFIGURATION EXAMPLE

### Hardware Configuration

### <Data Generation Device>

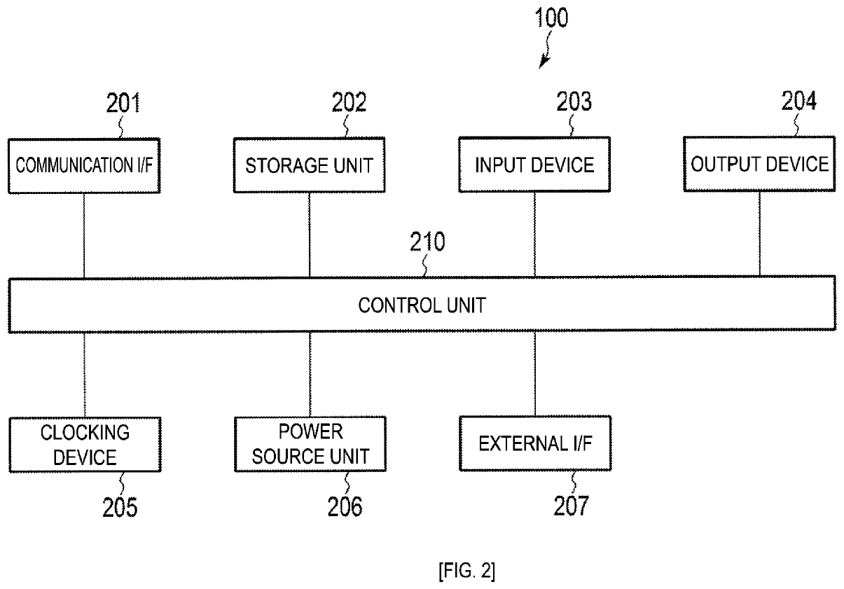

Next, there will be described an example of a hardware configuration of the data generation device 100 according to the present embodiment, referring to FIG. 2.

As illustrated in FIG. 2, the data generation device 100 according to the present embodiment includes a computer in which a communication interface 201, a storage unit 202, an input device 203, an output device 204, a clocking device 205, a power source unit 206, and an external interface 207 are electrically connected to a control unit 210. The data generation device 100 according to the present embodiment corresponds to the “data generation device” of the present invention. Note that, in FIG. 2, the communication interface and the external interface are described as “communication I/F” and “external I/F”, respectively.

The communication interface 201 is an interface for performing wired or wireless communication via a network, for example, a near-field wireless communication (e.g., Bluetooth (trade name)) module, a wired Local Area Network (LAN) module, or a wireless LAN module. The communication interface 201 is an interface for connecting the data generation device 100 to an external device (e.g., a computer, a communication apparatus on a network; the biometric data measurement device 120 and the medical professional terminal 110 in the example of FIG. 1). The communication interface 201, being controlled by the control unit 210, is intended to receive the biometric data from the biometric data measurement device 120, for example, and transmit the generated medical professional assistance data or limited medical professional assistance data to the medical professional terminal 110. In addition, the data generation device 100 may use the communication interface 201 to display the user-oriented data and/or the medical professional assistance data when storing the data in an external server or the like.

The storage unit 202 is a medium on which a computer or another device or a machine and the like accumulates information such as a recorded program using electric, magnetic, optical, mechanical, or chemical effects so that information such as the program can be read therefrom. The storage unit 202 may be an auxiliary storage device such as a hard disk drive or a solid-state drive, and may include a storage device connected via a network, as in the case of storing a part of the data in the server via the communication interface 201. The storage unit 202 stores the biometric data acquired from the biometric data measurement device 120. Furthermore, the storage unit 202 may store the user-oriented data, the medical professional assistance data, and/or the limited medical professional assistance data (encrypted medical professional assistance data) calculated by the control unit 210. In addition, the storage unit 202 may include a portable storage medium such as a memory card or a USB memory.

In addition, the storage unit 202 has stored therein an execution program to be executed by the control unit 210 for determining whether or not an anomaly exists based on the acquired biometric data, and generating the user-oriented data and/or the medical professional assistance data. Furthermore, the storage unit 202 also stores an execution program for presenting the recommendation data and/or the limited medical professional assistance data (encrypted medical professional assistance data).

Furthermore, as for the biometric data acquired by the biometric data measurement device 120, all the biometric data may be stored in the storage unit 202 over a predetermined time period. The predetermined time period is, for example, from the when the biometric data has been determined to indicate an anomaly to the time of receiving medical examination by the medical professional. In addition, the storage unit 202 may include biometric data before determination by the control unit 210, in which case the user's biometric state can be grasped by the progress data before the biometric data indicates an anomaly.

Moreover, the storage unit 202 may store one or more of user-related medical data and the lifestyle data, in a manner incorporated in the medical professional assistance data. The medical data is a type of data related to medical activities, for example, medicine ingestion data, allergy data, medical chart data, and medication record data. The lifestyle data, which is a type of data that may affect the biometric data related to the user's lifestyle, includes, for example, step-count data, walking distance data, jogging distance data, meal data, frequency and amount of alcohol drinking, frequency and amount of smoking cigarettes.

The input device 203 is a device configured to accept inputs, such as a touch panel, a physical button, a mouse, or a keyboard, for example. The output device 204 is a device such as a display, a speaker or a printer, for example, configured to output information via display, sound, paper media, or light. The touch panel, which can receive information input by touching the display panel with a finger, a pen, or the like, and also display thereon the button to be touched and other information, corresponds to both the input device 203 and the output device 204. In addition, the data generation device 100 may use the printer not as the output device 204 but as an external device via the communication interface 201 or the external interface 207. The output device 204 according to the present embodiment includes the “display unit” of the present invention.

The clocking device 205, which is a device configured to measure the time, can measure the date and time. For example, the clocking device 205, which may be a clock including a calendar, passes information of the current year and month and/or date and time to the control unit 210.

The power source unit 206 may be of any type as long as it can supply power such as, for example, rechargeable secondary battery or an AC power source obtainable from a normal electric outlet. The power source unit 206 supplies power to each component mounted on the main body of the data generation device 100. The power source unit 206 supplies power to the communication interface 201, the storage unit 202, the input device 203, the output device 204, the clocking device 205, the external interface 207, and the control unit 210, for example.

The external interface 207, which is used for mediating between the main body of the data generation device 100 and the outside, is a USB port, for example, and is an interface for connecting to an external device (e.g., memory, communication apparatus, biometric data measurement device). The external interface 207 is an interface for connecting to an external device such as, for example, an electrocardiograph, a sphygmomanometer, a pedometer, an activity meter, an acceleration sensor, and/or a printer.

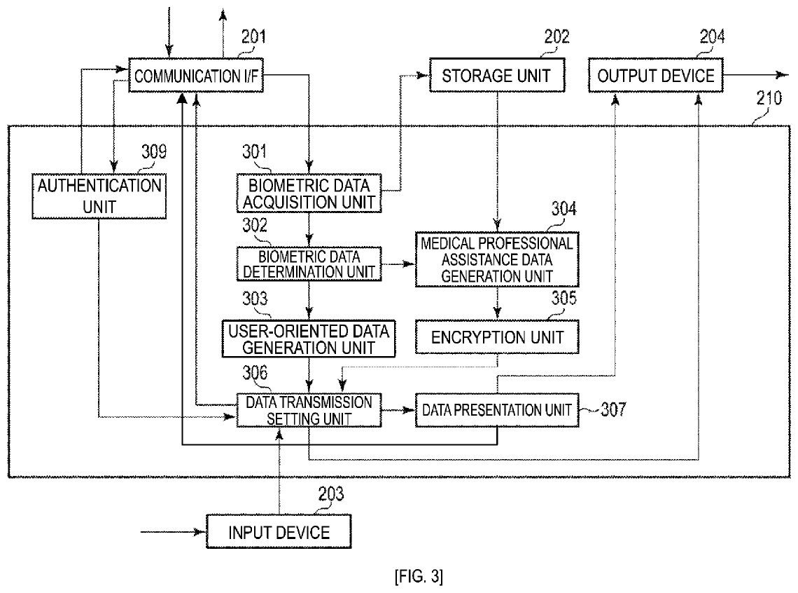

The control unit 210, includes a Central Processing Unit (CPU), a Random Access Memory (RAM), a Read Only Memory (ROM), and the like, and controls each component in accordance with the information processing. An execution program for generating the user-oriented data and/or the medical professional assistance data based on the acquired biometric data is stored in the storage unit 202, and the control unit 210 loads the execution program from the storage unit 202 to perform processing. Details of the control unit 210 will be described, referring to FIG. 3.

Software Configuration

### <Data Generation Device 100>

Next, there will be described an example of a software configuration of the data generation device 100 according to the present embodiment, referring to FIG. 3. FIG. 3 illustrates a software configuration for executing a program to be executed by the control unit 210 of the data generation device 100 for acquiring biometric data and determining whether or not the biometric data indicates an anomaly and, in a case where an anomaly exists, generating user-oriented data and medical professional assistance data.

When executing a required program, the control unit 210 of the data generation device 100 determines whether or not an anomaly exists based on the acquired biometric data stored in the storage unit 202 when executing the required program and, in a case where an anomaly exists, deploys the execution program for generating the user-oriented data and the medical professional assistance data on the RAM. Subsequently, the control unit 210 interprets and executes, by the CPU, the execution program deployed on the RAM and controls each component. As thus illustrated in FIG. 3, the data generation device 100 according to the present embodiment includes a biometric data acquisition unit 301, a biometric data determination unit 302, a user-oriented data generation unit 303, a medical professional assistance data generation unit 304, an encryption unit 305, the data transmission setting unit 306, a data presentation unit 307, and an authentication unit 309. In the present embodiment, the medical professional assistance data generation unit 304, the encryption unit 305, the data transmission setting unit 306, and the authentication unit 309 form the “access control unit” of the present invention. Additionally, in the present embodiment, the medical professional assistance data generation unit 304 and the data transmission setting unit 306 correspond to the “access destination-specifying unit” of the present invention.

The biometric data acquisition unit 301 acquires the biometric data of the user from an external biometric data measurement device 120 or the like via the communication interface 201. The biometric data acquisition unit 301 acquires the biometric data measured by the external biometric data measurement device 120, for example, at every prescribed time period via the communication interface 201. Although the prescribed time period is every one hour, for example, the biometric data acquisition unit 301 may acquire the biometric data with a shorter time period such as every minute, or a longer time period, such as a day. In addition, the biometric data may be continuously measured in terms of time by constantly connecting the external biometric data measurement device 120 via the communication interface 201 to acquire the biometric data continuously, or the biometric data may be acquired upon receiving transmission of data from the external biometric data measurement device 120 each time the external biometric data measurement device 120 performs measurement, or the user may request transmission of data to the external biometric data measurement device 120 using the input device 203 at an arbitrary timing to acquire the biometric data from the external biometric data measurement device 120. The manner of acquisition described above may be determined according to the type of biometric data and, for example, in case where step-count data, walking distance data, jogging distance data and the heart rate data are measured continuously in time, whereas the electrocardiographic data and the blood pressure data are measured at every prescribed time period, the biometric data acquisition unit 301 may acquire the measured data either continuously or at every prescribed time period in accordance with the type of data.

For example, in a case where the biometric data is electrocardiographic data, the biometric data acquisition unit 301 acquires, via the communication interface 201 as the electrocardiographic data, a potential difference signal indicating a potential difference between two electrodes output in time series from the signal processing circuit in the biometric data measurement device 120. Here, the electrocardiographic data is a waveform signal representing electrical activity of the heart. In a case where the biometric data is blood pressure data, the biometric data acquisition unit 301 acquires, via the communication interface 201, blood pressure data (data including systolic blood pressure (SBD) and diastolic blood pressure (DBP)) measured by the biometric data measurement device 120.

The biometric data acquired by the biometric data acquisition unit 301 is passed to the biometric data determination unit 302 and stored in the storage unit 202. The biometric data acquisition unit 301 may store all the acquired biometric data in the storage unit 202. In addition, the biometric data acquisition unit 301 may acquire medical data related to the user via the communication interface 201, and store it in the storage unit 202. Furthermore, the biometric data acquisition unit 301 may acquire lifestyle data that may affect the biometric data related to the lifestyle of the user via the communication interface 201, and store it in the storage unit 202. The medical data and the lifestyle data may be stored in the storage unit 202 to be incorporated in the medical professional assistance data, and the medical professional assistance data generation unit 304 may generate medical professional assistance data including such data.

Note that, in the present embodiment, although the sensor for detecting biometric data is mounted on the biometric data measurement device 120, the data generation device 100 may include such devices, from which the biometric data acquisition unit 301 may acquire biometric data.

The biometric data determination unit 302 determines whether or not the biometric data acquired by the biometric data acquisition unit 301 indicates an anomaly. The biometric data determination unit 302 determines, for example, a level indicating a degree of anomaly set for each type of biometric data, and determines that the biometric data indicates an anomaly in a case where the level is equal to or higher than a threshold value indicating an anomaly. The levels may differ in physical quantity and range of magnitude depending on the type of biometric data, and further the physical quantity and the range of magnitude may vary depending on the causes of the anomaly (e.g., type of disease).

For example, in case where the biometric data is electrocardiographic data, the electrocardiographic data indicates electrical excitation of the heart and temporal variation in the subsidence of the electrical excitation, and the electrical potential varies due to variation of an electric field generated on the body surface by the electrical excitation of the heart. The biometric data determination unit 302 determines that an anomaly exists in a case where the electrical potential of the electrocardiographic data and variation thereof is equal to or larger than a threshold value. More specifically, the biometric data determination unit 302 determines that an anomaly exists in a case where the electrocardiographic data indicates that (1) the number of pulses within a predetermined time period is equal to or larger than a first threshold value and the pulses appears earlier than peripheral cycles by the first threshold value or more (tachyarrhythmia), and (2) the number of pulses within a predetermined time period is less than a second threshold value and appearance of pulses is delayed than peripheral cycles by the second threshold value or more (bradyarrhythmia). For other electrocardiographic data, there may be, for example, anomaly in P-wave, anomaly in QRS, anomaly in ST, anomaly in T-wave, anomaly in PQ interval, anomaly in QRS interval, or anomaly in QT interval, and the biometric data determination unit 302 may determine whether or not such an anomaly has occurred. There are a large number of methods for determining anomalies in the electrocardiographic data depending on anomaly types, all of which can be determined from the electrocardiographic data, and therefore the biometric data determination unit 302 may perform determination using such techniques.

In a case where the biometric data is blood pressure data, the biometric data determination unit 302 determines that an anomaly exists in the blood pressure data when the numerical value of time series data including systolic blood pressure (SBP) and diastolic blood pressure (DBP) has changed by a threshold value or more within a predetermined time period. Additionally, in a case where the time period, in which the systolic blood pressure value is equal to or larger than a threshold value, is equal to or longer than a threshold time period, the biometric data determination unit 302 may determine that an anomaly exists in the blood pressure data. The blood pressure data also changes in various manners, the biometric data determination unit 302 may determine whether or not an anomaly exists in the blood pressure data according to variation of the value.

In a case where the biometric data is step-count data, the biometric data acquisition unit 301 acquires, per hour for example, the number of steps the user has walked in the time zone. For step-count data, the biometric data determination unit 302, referring also to the measurement time point, may determine that an anomaly exists in a case where there are very few steps (e.g., the number of steps per hour continuously being equal to or less than a threshold value for a predetermined number of times) even in day time, for example.

The user-oriented data generation unit 303 generates user-oriented data in a case where the biometric data determination unit 302 has determined that the biometric data indicates an anomaly. Here, the user-oriented data is a type of data that recommends the user to receive medical examination by a medical professional. The user-oriented data includes content that recommends the user to receive medical examination by a medical professional and, in a case where the biometric data determination unit 302 has determined that the electrocardiographic data indicates an anomaly, for example, the user-oriented data generation unit 303 generates a statement data, “Your pulse is disturbed. Please receive medical examination by a medical professional before the end of the day,” as the user-oriented data, for example. In addition, the user-oriented data generation unit 303 may change the content of the user-oriented data depending on the degree of anomaly determined by the biometric data determination unit 302. For example, in a case where the degree of anomaly is larger than a predetermined value (e.g., a value of the biometric data is equal to or larger than a threshold value), the user-oriented data generation unit 303 may generate user-oriented data including recommendation in a strong tone that the user is in an emergency situation, and may also generate data directed to the user's close relatives and/or caregiver, and data by which the user can be identified, separately from the medical professional assistance data described below. The above-described data are transmitted by the data transmission setting unit 306 to the user's close relatives and/or caregiver via the communication interface 201. In addition, the above-described data may be output to the output device 204 via the data transmission setting unit 306 and the data presentation unit 307, and notified to the user in a form including sound and/or light.

The medical professional assistance data generation unit 304 generates medical professional assistance data directed to a medical professional in a case where the biometric data determination unit 302 has determined that the biometric data indicates an anomaly. The medical professional assistance data includes medical expertise and also includes data for assisting the medical professional to perform appropriate medical examination and/or treatment by the medical professional for the user. The medical professional assistance data may include, in addition to detailed information of the biometric data, a plurality of pieces of information such as names or diagnostic information of diseases likely to be contracted including the name or diagnostic information of the disease that is most likely to be contracted from the detailed information. In addition, the medical professional assistance data may include biometric data related to an anomaly, or biometric data with a marker or the like applied to specify a part of the biometric data. The marker of the present embodiment corresponds to “anomaly-specifying data” of the present invention.

In addition, the medical professional assistance data generation unit 304 may incorporate, in the medical professional assistance data, not only the biometric data used by the biometric data determination unit 302 for determination, but also the biometric data acquired since the determination. For example, the biometric data acquisition unit 301 acquires, and stores in the storage unit 202, biometric data since the determination by the biometric data determination unit 302 until the user receives medical examination by the medical professional, and the medical professional assistance data generation unit 304 acquires, and incorporates in the medical professional assistance data, the biometric data from the storage unit 202. In addition, the medical professional assistance data generation unit 304 may generate medical professional assistance data which will probably turn out to be useful for medical examination and diagnosis by the medical professional, based on the biometric data, acquired from the storage unit 202, which is the data up to immediately before the medical examination.

Furthermore, the medical professional assistance data generation unit 304 may perform certain encoding on the medical professional assistance data to generate data. The medical professional assistance data generation unit 304 may convert the medical professional assistance data into a two-dimensional code, i.e., generate data (image data) described in a two-dimensional code as the limited medical professional assistance data. The encoding may be performed by any unit other than the medical professional assistance data generation unit 304 such as, for example, the encryption unit 305. The content of the two-dimensional code cannot be read at a glance, and therefore confidentiality can be easily preserved. Furthermore, the encryption unit 305 described below may be set to encrypt the two-dimensional code.

The encryption unit 305 encrypts medical professional assistance data into limited medical professional assistance data so that only a medical professional is allowed to view the content of the medical professional assistance data generated by the medical professional assistance data generation unit 304. Although it is almost impossible for anyone other than a medical professional viewing the medical expertise to comprehend the content thereof, it is possible that someone, including the user, other than a medical professional viewing the medical professional assistance data including detailed data related to the biometric data may erroneously recognize the content thereof, which is likely to bring about more demerits than merits. In addition, it is necessary to conform to a social agreement, if any, that it is desirable to provide the user with explanation of the biometric data, and provide appropriate explanation in a case of a disease notification thereof by a medical professional, whereas it is not desirable for the user to view the biometric data in a state in which the user have not received explanation by a medical professional. In addition, leaving the medical professional assistance data related to the biometric data of a certain user in a state viewable by a large number of unspecified persons may lead to leakage of personal information and therefore is undesirable.

The encryption unit 305 performs a process of limiting the knowability of the medical professional assistance data to the medical professional assistance data. The limiting process may be of any type as long as it can render the medical professional assistance data impossible to be read by a third party when displayed or printed and decryptable by a predetermined algorithm, and therefore the medical professional assistance data may be encrypted by conventional encryption techniques using an encryption theory, the medical professional assistance data may be information encoded (data compression) with a certain code, for example encoded in a two-dimensional code. Here, both the medical professional assistance data encoded using data compression and the medical professional assistance data encrypted using an encryption theory are referred to as limited medical professional assistance data. Any encoding algorithm and any encryption technique may be used by the encryption unit 305, and for example, there are data compression schemes such as ZIP, LHA, and encryption schemes such as RSA encryption or AES encryption. The encryption unit 305 encrypts the medical professional assistance data with the medical professional using a password, a public key, or a private key, which is preliminarily known. The medical professional may use the medical professional terminal 110 to decrypt the encrypted medical professional assistance data using the password or key. Although the medical professional assistance data encrypted based on data compression cannot be decrypted without decoding, the algorithm is known and therefore it is desirable to set a password when performing data compression on the medical professional assistance data. The encoding and/or encryption performed by the medical professional assistance data generation unit 304 and/or the encryption unit 305 are intended to limit the knowability about the medical professional assistance data, whereas the decryption using a password or a key is intended to release the limitation on the knowability. In other words, the data generation device 100 controls the knowability about the medical professional assistance data by the limitation and release.

In addition, the encryption unit 305 may encode the medical professional assistance data to further encrypt the same. For example, the encryption unit 305 may encode the medical professional assistance data into two-dimensional code and subsequently encrypt the same. In order to decrypt the encrypted medical professional assistance data, software is used that acquires a password and an image of the two-dimensional code for resolving the encryption and further decrypts the data. The password has been preliminarily distributed to a medical professional, or the password generated at the time of encryption is transmitted to the medical professional. In addition, the decryption software may be preliminarily distributed to the medical professional, or installed on the medical professional terminal 110.

Furthermore, the encryption unit 305 may change the encryption scheme depending on the medical professional or the medical institution to which the medical professional belongs. Although any medical professional can decode and/or decrypt the limited medical professional assistance data (encrypted medical professional assistance data) and read the content of medical professional assistance data provided that software and/or password available to any medical professional is used, there may be a case where it is desirable to allow only doctors belonging to a specified medical institutions or specified doctors to know the medical professional assistance data, based on the content of the biometric data or the medical professional assistance data, the user's doctor, or social request. Here, user operation, initial setting of the data generation device 100, and the encryption unit 305 of the data generation device 100 may generate limited medical professional assistance data so that only a specified medical professional can know the content of the medical professional assistance data, depending on the type of biometric data acquired by the biometric data acquisition unit 301.

For example, generating a one-time password by the encryption unit 305, using the one-time password to encode and/or encrypt the medical professional assistance data, and further notifying only a specified medical professional of the one-time password, allows the only specified medical professional to read the medical professional assistance data from the access-enabling data using the one-time password. The specified medical professional is a medical professional currently performing medical examination, for example, and notifying only the medical professional of the one-time password allows the only medical professional performing medical examination to read the content of the medical professional assistance data of the user. The one-time password may be presented or transmitted with any method as long as only a specified medical professional can know it, and is notified by a message to the telephone number or mail address of the medical professional, for example. In addition, the user may cause the display unit of the data generation device 100 to display the one-time password, and show the medical professional the screen of the display unit having the one-time password displayed thereon. The one-time password according to the present embodiment corresponds to the “access-enabling data” of the present invention. In addition, the medical professional assistance data encoded and/or encrypted using the one-time password according to the present embodiment corresponds to the “limited medical professional assistance data” of the present invention.

The data transmission setting unit 306 performs a setting of how to present or transmit respective pieces of the access-enabling data to the user and the medical professional, and passes each data to an appropriate device, when generating the user-oriented data and the medical professional assistance data, as well as the access-enabling data. The data transmission setting unit 306 sets the user-oriented data to be displayed on the display which is the output device 204 via the data presentation unit 307, for example. In addition, the data transmission setting unit 306, upon accepting a request from the medical professional terminal 110 to transmit the medical professional assistance data, for example, may perform a setting to display, on a display which is the output device 204, the name of the medical professional logging to the medical professional terminal 110, the fact that a request for transmission is being received, and a user interface (e.g., “OK” button) that allows the user to perform an operation that permits transmission, and also a setting to transmit the limited medical professional assistance data via the communication interface 201 according to the near-field wireless communication method when the authentication unit 309 authenticates the medical professional terminal 110 as the transmission destination by permission operation performed by the user. According to the above-described setting, the limited medical professional assistance data is transmitted after the user has confirmed that the person using the medical professional terminal 110 is the medical professional performing medical examination, and the authentication unit 309 has authenticated that the medical professional terminal 110 is appropriate as the transmission destination, and therefore it is possible to transmit the limited medical professional assistance data reliably to a desired medical professional. The near-field wireless communication method is not limited to some specific scheme, and may be any communication scheme, for example, Bluetooth (trade name) or Near Field Communication (NFC).

In addition, the data transmission setting unit 306 passes an instruction for displaying or printing to present the user-oriented data and the limited medical professional assistance data to the data presentation unit 307 together with the user-oriented data and the limited medical professional assistance data, in a case where the medical professional assistance data has been encoded and/or encrypted into the limited medical professional assistance data using a two-dimensional code, for example. The data presentation unit 307 displays the limited medical professional assistance data together with the user-oriented data on the display which is the output device 204 of the data generation device 100, or prints out paper media or the like from the printer using the output device 204 or the external interface 207. The user can read a message (text data), as the user-oriented data, that recommends to receive medical examination by a medical professional, by a display on the output device 204 or a printed material. On this occasion, with the limited medical professional assistance data also being displayed or printed together with the user-oriented data, although the user can recognize that the limited medical professional assistance data is displayed or printed thereon, the limited medical professional assistance data has been encoded and encrypted with the two-dimensional code, preventing the user from reading the content thereof, and importing the two-dimensional code by another terminal belonging to the user does not allow for decryption and/or decoding, thereby preventing the user from knowing the content of the limited medical professional data. On the other hand, the medical professional can import the limited medical professional assistance data displayed or printed on the output device 204 into the medical professional terminal 110 with an image acquisition unit of the medical professional terminal 110 or an image acquisition device connected to the medical professional terminal 110, and decrypt and/or decode the medical professional assistance data (limited medical professional assistance data) encrypted by a password or a cryptographic key and know the content thereof.

In addition, the data presentation unit 307 may not only present the user-oriented data and the limited medical professional assistance data, but also prompt the user to notify the medical professional of existence of the limited medical professional assistance data, or pass the limited medical professional assistance data to the medical professional.

The authentication unit 309 performs mutual authentication with a biometric data measurement device 120 that transmits the biometric data to the data generation device 100 via the communication interface 201, and upon authenticating that the biometric data measurement device 120 is the device from which the data generation device 100 is supposed to acquire the biometric data, enables the data generation device 100 to receive the biometric data from the biometric data measurement device 120. In addition, the authentication unit 309 may perform mutual authentication with the medical professional terminal 110 which is the counterpart of transmitting the medical professional assistance data (including the limited medical professional assistance data) and/or the access-enabling data and, upon authenticating that the medical professional terminal 110 is the device to which the biological data is supposed to be transmitted from the data generation device 100, enables transmission of the medical professional assistance data and/or the access-enabling data to the authenticated medical professional terminal 110. In addition, an interface for accepting user permission of association between the specific medical professional terminal 110 and the data generation device 100 is preliminarily displayed on a display unit such as a display device. Subsequently, with a permission by the user via the interface, and upon the authentication unit 309 of the data generation device 100 authenticating that the medical professional terminal 110 has been preliminarily associated, the medical professional assistance data, the limited medical professional assistance data, and/or the user-oriented data may be transmitted from the data generation device 100 to the medical professional terminal 110, in response to a request from the medical professional terminal 110. As has been described above, by associating the data generation device 100 and the medical professional terminal 110 with preliminarily acquired user permission, when receiving medical examination, in response to a request from the medical professional terminal 110, the data presentation unit 307 is allowed to display the above-mentioned data on a display and the like or transmit an instruction for printing the above-mentioned data to a printer or the like external to the device, without requiring an individual permission or operation of the user.

Other

Operation of the data generation device 100 will be explained in detail in the operation example described below. Note that, in the present embodiment, the control unit 210 of the data generation device 100 may be realized by a general-purpose CPU. However, a part or all of the above-mentioned operations (functions) may be realized by one or more dedicated processors. In addition, the data generation device 100 is not limited to a case of realization by a single-device, but may be configured to include a plurality of devices, and a part of the processing may be performed by a server. In addition, components of the data generation device 100 may be omitted, substituted, or added as appropriate in accordance with embodiments.

### Operation Example: Overall

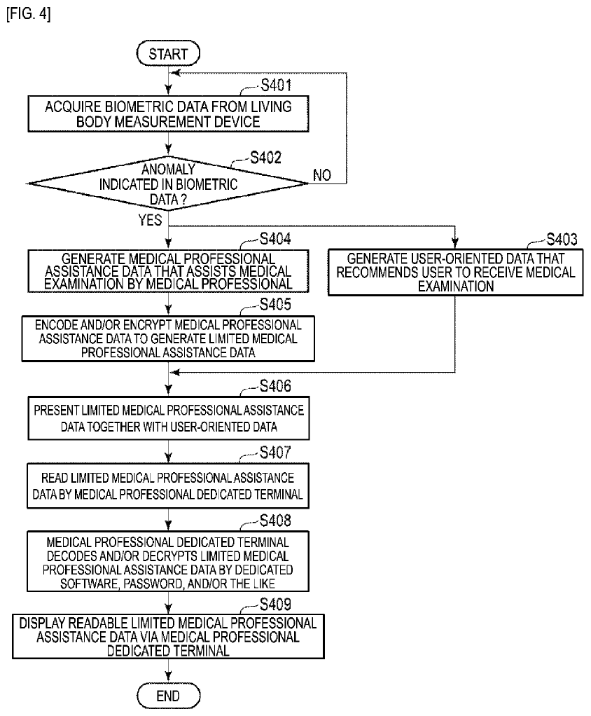

Next, there will be described an outline of an operation of the data generation device 100, referring to FIG. 4.

FIG. 4 is a flowchart illustrating an example of a processing procedure (data generation method) of the data generation device 100. Note that the processing procedure described below is merely an example, and each process may be changed to the extent possible. Further, in the processing procedure described below, steps can be omitted, substituted, and added in accordance with the embodiment as appropriate. Note that S407 and subsequent steps are not the processing procedure by the data generation device 100, but are the processing procedure by the medical professional terminal 110 described for reference.

Activation

First, the user activates, via the input device 203, the data generation device 100, which in turn accepts input such as settings. The control unit 210 of the data generation device 100 performs the process according to the following processing procedure.

(Step S401)

At step S401, the control unit 210, operating as the biometric data acquisition unit 301, acquires, via the communication interface 201, biometric data of the user from the biometric data measurement device 120 authenticated by the authentication unit 309. The biometric data may be acquired by the biometric data acquisition unit 301, and stored in the storage unit 202 from the biometric data acquisition unit 301 in sequence. The biometric data may be stored in the storage unit 202 in accordance with the date-and-time data of the clocking device 205, or the biometric data may be stored in the storage unit 202 together with the date-and-time data provided by a clocking device of the biometric data measurement device 120 that has acquired the biometric data.

(Step S402)

At step S402, the control unit 210, operating as the biometric data determination unit 302, determines whether or not the biometric data acquired at step S401 indicates an anomaly. The timing of determination generally differs depending on the type of biometric data or the measurement method. For example, in a case where the biometric data is electrocardiographic data, which may vary in seconds, although it is desirable to determine whether or not biometric data indicates anomaly at an interval of seconds or tens of seconds, determination may be performed at a time interval or timing which is acceptable by the acquisition scheme and/or measurement method in a case where the measurement device collectively acquires measured biometric data after completion of measurement, or in a case where it is difficult to measure electrocardiographic data continuously in time (e.g., in seconds). In addition, with the biometric data measurement device 120 starting measurement upon instruction by the user to start measurement, the biometric data acquisition unit 301 may acquire biometric data of the user simultaneously with completion of measurement or start of measurement, and the biometric data determination unit 302 may determine whether or not the biometric data indicates an anomaly.

In a case where the biometric data does not indicate any anomaly at step S402, the process returns to step S401, at which the biometric data acquisition unit 301 acquires the biometric data. As long as there exists yet-to-be-determined biometric data that can be determined by the biometric data determination unit 302, the process returns to step S401 to repeat determination.

(Step S403)

At step S403, the control unit 210, operating as the user-oriented data generation unit 303, generates user-oriented data that recommends the user to receive medical examination by a medical professional, because the biometric data indicates an anomaly.

(Step S404)

At step S404, the control unit 210, operating as the medical professional assistance data generation unit 304, generates medical professional assistance data that assists the medical examination performed by the medical professional on the user, because the biometric data indicates an anomaly. Specifically, in a case where there is a symptom indicating arrhythmia in a part of the electrocardiographic data in a single measurement (e.g., 30 seconds), the medical professional assistance data may include raw data, a marker applied to a part (time point) of the raw data indicating possibility of arrhythmia, type of arrhythmia and name of disease that can be estimated therefrom, or examination recommended to be subsequently performed.

(Step S405)

At step S405, the control unit 210, operating as the encryption unit 305, encodes and/or encrypts the medical professional assistance data to generate the limited medical professional assistance data.

(Step S406)

At step S406, the control unit 210, operating as the data transmission setting unit 306 and the data presentation unit 307, displays the encoded and/or encrypted medical professional assistance data (i.e., limited medical professional assistance data) and the user-oriented data on the output device 204, or prints them.

Step S407 and subsequent steps are not processes performed by the data generation device 100, but are processing procedures by the medical professional terminal 110, for example.

(Step S407)

At step S407, the medical professional terminal 110 reads the limited medical professional assistance data presented at step S406.

(Step S408)

At step S408, the medical professional terminal 110 decodes and/or decrypts the limited medical professional assistance data using dedicated software and/or a password for decrypting the limited medical professional assistance data, and converts the content of the medical professional assistance data into a format that can be read by a medical professional.

(Step S409)

At step S409, the medical professional terminal 110 presents, on a display or the like, the medical professional assistance data converted from the limited medical professional assistance data at step S408 to be knowable by the medical professional.

### Actions and Effects

As has been described above, the data generation device 100 of the present embodiment generates user-oriented data and medical professional assistance data for a medical professional at steps S403 and S404, in a case where the biometric data determination unit 302 has determined that the biometric data indicates an anomaly at step S402. Subsequently, at step S405, the medical professional assistance data is encrypted to generate limited medical professional assistance data so that only a medical professional can refer to the content thereof (or briefly speaking, medical professional assistance data is encoded, for example). As a result, the data generation device 100 can generate medical professional assistance data including medical expertise for assisting the medical professional to apply an appropriate medical treatment to the user, and generate and set the medical professional assistance data into limited medical professional assistance data which cannot be viewed by anyone other than a medical professional.

Furthermore, the data generation device 100 can enable the user to recognize, with the user-oriented data, the necessity to receive medical examination and/or medical practice by a medical professional by presenting the limited medical professional assistance data together with the user-oriented data to the user at step S406, and, with the limited medical professional assistance data being presented at the same time, it can be expected that appropriate medical examination and medical practice can be received by passing the data to the medical professional. In addition, from the viewpoint of the medical professional, it can be assumed that the limited medical professional assistance data that can be read by the medical professional terminal 110 at steps S407, S408, and S409 allows for grasping in detail the reason for receiving medical consultation, which has conventionally been acquired from the user by medical interview or the like, and also it becomes likely to quickly find the cause of the anomaly indicated by the biometric data. In addition, it becomes possible for the user to visit the medical institution within a short time since the biometric data has indicated an anomaly, and therefore the user can be applied medical examination and treatment at an early stage, thereby increasing the possibility of treating the user before the user's symptoms proceeds to a serious stage. In addition, it turns out that medical examination and treatment can be applied at an early stage, making it more likely to provide a wider range of selecting treatment methods, and therefore it becomes possible to apply safer and more appropriate medical practice to the user.

### Second Embodiment

### [Overview]

The data generation device of a second embodiment differs from the data generation device of the first embodiment in that the medical professional assistance data or the limited medical professional assistance data are uploaded to a server so that the medical professional terminal acquires the limited medical professional assistance data and/or medical professional assistance data from the server. There are cases where the data generation device of the second embodiment transmits the medical professional assistance data to the server as it is, or transmits the limited medical professional assistance data to the server.

The following explanation of the second embodiment will describe only different points from the first embodiment.

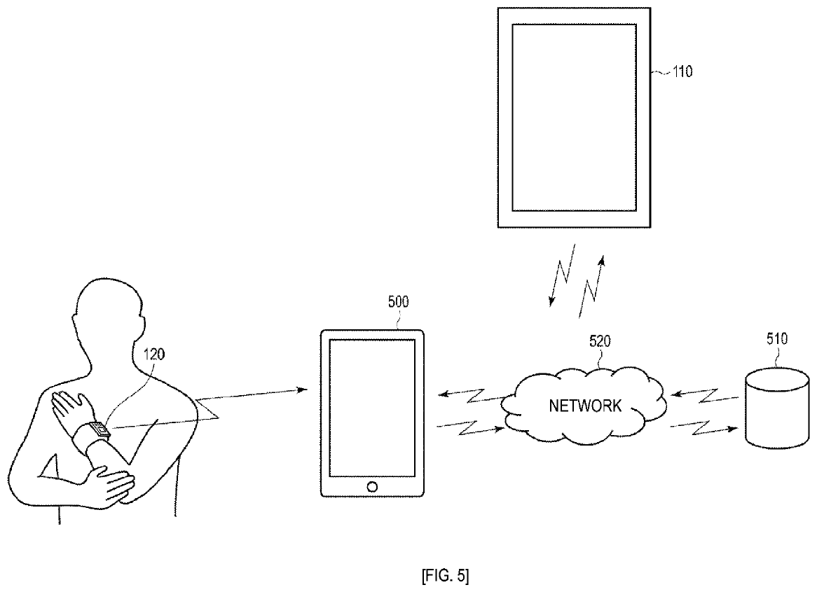

First, an outline of the data generation device according to the second embodiment of the present invention will be described, referring to FIG. 5. FIG. 5 schematically illustrates an example of a data generation device 500, a medical professional terminal 110, the biometric data measurement device 120, a server 510, and a network 520 according to an example described in the overview. The medical professional assistance data is stored in the server 510 without being encrypted, to which an access right is set to a medical professional. On this occasion, the address of the server 510 having the medical professional assistance data stored therein is displayed as access-enabling data on the display unit, which is the output device 204 of the data generation device 500, or printed from a printer external to the device, so that the displayed or printed address is presented to a medical professional. A medical professional who has viewed the address inputs the address to the medical professional terminal 110, or acquires the address via an image acquisition unit included in the medical professional terminal 110 or an image acquisition device connected to the medical professional terminal 110. Accessing, by the medical professional terminal 110, a specific area of the server 510 specified by the address allows the medical professional to view and know the content of the medical professional assistance data. On this occasion, authenticating that the person using the medical professional terminal 110 is a medical professional is performed by a data request from software dedicated to a medical professional or by using an ID and/or password of the system dedicated to a medical professional.

In addition, the server 510 may have stored therein the user-oriented data in relation to the medical professional assistance data. In such a case, the server 510 is set so that the user, in addition to the medical professional, can access the user-oriented data related to the user. The user-oriented data may be set to allow the user to log in and access (the medical professional can also access) the server 510 using an account and a password.

Transmitting the user-oriented data and the medical professional assistance data to the server by the data generation device 500 eliminates the necessity of storing the data in the data generation device 500 and, since the medical professional assistance data stored in the server can be accessed by only a medical professional, it is possible to manage data unitarily and securely while preserving confidentiality of the data. In addition, the data generation device 500 allows the user and/or the medical professional to acquire the user-oriented data and the medical professional assistance data by using a terminal that can access the server 510, even in a case where the terminal of the user and/or the medical professional fails or is lost or the like.

Although a case of storing non-encrypted medical professional assistance data in the server 510 has been described, it is also possible to store encoded and/or encrypted medical professional assistance data (limited medical professional assistance data) in a specific area of the server 510. In such a case, an encryption key or software for decrypting the encoded and/or encrypted medical professional assistance data is used as an access-enabling tool, in addition to the address as an access enabling tool. As in the above-described manner, storing the limited medical professional assistance data in the server 510 allows for strictly limiting the knowability of the medical professional assistance data, making it reliably confidential.

As has been described above, according to the data generation device of the present embodiment, the data generation device of the present embodiment allows for recommending a user to receive medical examination by a medical professional triggered by the biometric data indicating an anomaly, as well as providing detailed medical data with regard to the biometric data indicating an anomaly of the user to the medical professional performing medical examination of the user based on the same biometric data, and therefore it becomes more likely that the user can receive appropriate medical examination and treatment from the doctor and also it becomes possible to manage data unitarily and securely. In addition, according to the present embodiment, the user and/or the medical professional can acquire the user-oriented data and the medical professional assistance data by using a terminal that can access the server 510, even in a case where the terminal of the user and/or the medical professional fails or is lost or the like. Other effects are similar to those by the first embodiment.

### Configuration Example

### Hardware Configuration

### <Data Generation Device>

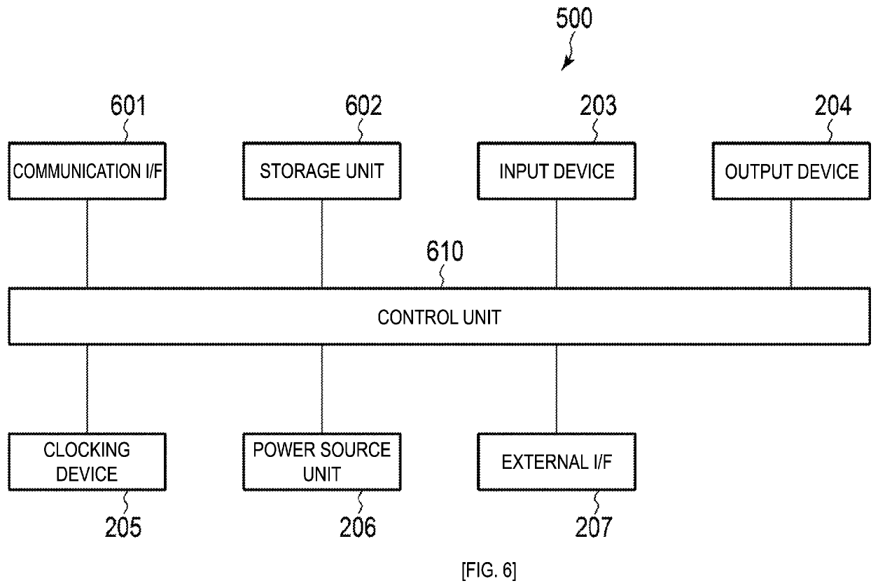

Next, there will be described an example of a hardware configuration of the data generation device 500 according to the present embodiment, referring to FIG. 6.

As illustrated in FIG. 6, the data generation device 500 according to the present embodiment includes a computer in which a communication interface 601, a storage unit 602, the input device 203, the output device 204, the clocking device 205, the power source unit 206, and an external interface 207, are electrically connected to a control unit 610. The data generation device 500 according to the present embodiment corresponds to the “data generation device” of the present invention. Note that, in FIG. 6, the communication interface and the external interface are expressed as “communication I/F” and “external I/F”, respectively.

The communication interface 601 is an interface for performing wired or wireless communication via a network, for example, a near-field wireless communication (e.g., Bluetooth (trade name)) module, a wired Local Area Network (LAN) module, or a wireless LAN module. The communication interface 601 is an interface for connecting the data generation device 500 to an external device (e.g., a computer, a communication apparatus on a network; in the example of FIG. 5, a biometric data measurement device 120, a medical professional server 510). The communication interface 601, being controlled by the control unit 610, is intended to receive the biometric data from the biometric data measurement device 120, for example, and transmit the generated medical professional assistance data to the medical professional terminal 110. Moreover, the communication interface 601 is intended for uploading the user-oriented data and the medical professional assistance data to the server 510 via the network 520, and download the user-oriented data and/or the medical professional assistance data from the server 510 when necessary. In addition, the data generation device 500 connects to the control unit 610 using the communication interface 601 without downloading the user-oriented data and/or the medical professional assistance data, when simply viewing the data.

The communication over the network is usually wireless but may be wired. Note that the network may be an inter-network including the Internet, or another type of network such as an intra-hospital LAN, or one-to-one communication using a Universal Serial Bus (USB) cable or the like. The communication interface 601 may include a micro-USB connector.

The storage unit 602 is a medium on which a computer or another device, or a machine and the like accumulates information such as a recorded program using electric, magnetic, optical, mechanical, or chemical effects so that the information such as the program can be read therefrom. In addition, the storage unit 602 may store data indicating the address of the server 510 in a case where the user-oriented data and/or the medical professional assistance data are stored in the server 510. In addition, the storage unit 602 may include a portable storage medium such as a memory card or a USB memory.

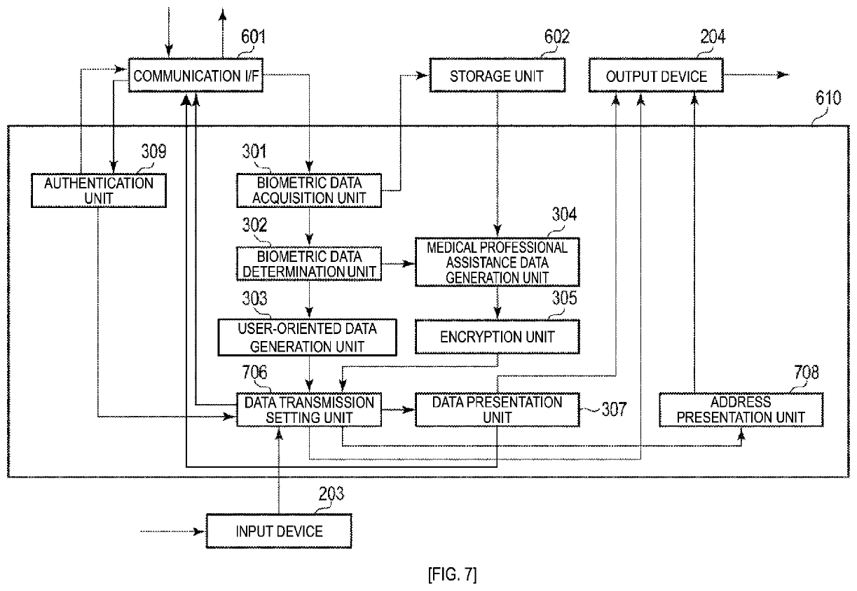

The control unit 610, includes a Central Processing Unit (CPU), a Random Access Memory (RAM), or a Read Only Memory (ROM), and controls each component in accordance with the information processing. An execution program for generating the user-oriented data and/or the medical professional assistance data based on the acquired biometric data is stored in the storage unit 602, and the control unit 610 loads the execution program from the storage unit 602 to perform processing. Details of the control unit 610 will be described, referring to FIG. 7.

Software Configuration

### <Data Generation Device 500>

Next, there will be described an example of a software configuration of the data generation device 500 according to the present embodiment, referring to FIG. 7. FIG. 7 illustrates a software configuration for executing a program to be executed by the control unit 610 of the data generation device 500 for acquiring biometric data and determining whether or not the biometric data indicates an anomaly and, in a case where an anomaly exists, generating user-oriented data and medical professional assistance data.

When executing a required program, the control unit 610 of the data generation device 500 determines whether or not an anomaly exists based on the acquired biometric data stored in the storage unit 602 when executing the required program and, in a case where an anomaly exists, deploys the execution program for generating the user-oriented data and the medical professional assistance data on the RAM. Subsequently, the control unit 610 interprets and executes, by the CPU, the execution program deployed on the RAM and controls each component. As thus illustrated in FIG. 7, the data generation device 500 according to the present embodiment includes the biometric data acquisition unit 301, the biometric data determination unit 302, the user-oriented data generation unit 303, the medical professional assistance data generation unit 304, the encryption unit 305, a data transmission setting unit 706, the data presentation unit 307, an address presentation unit 708, and an authentication unit 309. In the present embodiment, the medical professional assistance data generation unit 304, the encryption unit 305, the data transmission setting unit 706, the address presentation unit 708, and the authentication unit 309 form the “access control unit” of the present invention.

The biometric data acquired by the biometric data acquisition unit 301 is passed to the biometric data determination unit 302 and stored in the storage unit 602. The biometric data acquisition unit 301 may store, in the storage unit 602, all the biometric data prior to occurrence of an anomaly. In addition, the biometric data acquisition unit 301 may acquire medical data related to the user via the communication interface 601, and store it in the storage unit 602. Furthermore, the biometric data acquisition unit 301 may acquire lifestyle data that may affect the lifestyle data associated with lifestyle related to the user via the communication interface 601, and store it in the storage unit 602. The medical data and the lifestyle data may be stored in the storage unit 602 to be incorporated in the medical professional assistance data, and the medical professional assistance data generation unit 304 may generate medical professional assistance data including such data.

Note that, although the external sensor for detecting biometric data is mounted on the biometric data measurement device 120, the data generation device 500 may include such devices, from which the biometric data acquisition unit 301 may acquire biometric data.

The user-oriented data is output to the output device 204 via the data presentation unit 307 by the data transmission setting unit 706 and presented to the user.

In addition, the medical professional assistance data generation unit 304 may incorporate, in the medical professional assistance data, not only the biometric data used by the biometric data determination unit 302 for determination, but also the biometric data acquired since the determination. For example, the biometric data acquisition unit 301 acquires, and stores in the storage unit 602, biometric data since the determination by the biometric data determination unit 302 until the user receives medical examination by the medical professional, and the medical professional assistance data generation unit 304 acquires, and incorporates in the medical professional assistance data, the biometric data from the storage unit 602. In addition, the medical professional assistance data generation unit 304 may generate medical professional assistance data which will probably turn out to be useful for medical examination and diagnosis by the medical professional, based on the biometric data, acquired from the storage unit 602, which is the data up to immediately before the medical examination.

Furthermore, the medical professional assistance data generation unit 304 may be set so that the encryption unit 305 described below encrypts the two-dimensional code, or accesses a file in the server 510 using a two-dimensional code.

The data transmission setting unit 706 performs a setting of how to transmit the user-oriented data and the medical professional assistance data to the user and the medical professional, and passes each data to an appropriate device.

The data transmission setting unit 706 transmits the user-oriented data and the (encrypted or unencrypted) medical professional assistance data, for example, to the server 510 via the communication interface 601. The server 510 has preliminarily prepared therein, for example, an area for storing the user-oriented data and an area for storing the medical professional assistance data, and the data transmission setting unit 706 are sets such that each data is stored in the corresponding area. In addition, the area for storing the user-oriented data is set to be accessible by at least the user, and the medical professional assistance data is set to be accessible by only a medical professional. Transmitting the user-oriented data and the medical professional assistance data to the server 510 eliminates the necessity of storing the data in the data generation device 500 and, since the medical professional assistance data stored in the server 510 can be accessed by only a medical professional, it is possible to manage data unitarily and securely while preserving confidentiality of the data. In addition, the data generation device 500 allows the user and/or the medical professional to acquire the user-oriented data and the medical professional assistance data by using a terminal that can access the server 510, even in a case where the terminal of the user and/or the medical professional fails or is lost or the like.

Transmission of the data may be performed based on the user's instruction such that, for example, the user touches the display of an interface displayed on the input device 203, and performs a setting that causes the data transmission setting unit 706 to transmit the medical professional assistance data, and transmits to the server 510 via the communication interface 601. The data transmission setting unit 706 sets the medical professional assistance data to be transmitted according to the near-field wireless communication method, and the medical professional assistance data is transmitted after the authentication unit 309 has authenticated that the transmission destination is the medical professional terminal 110, and therefore it is possible to transmit the medical professional assistance data reliably to a desired medical professional. The near-field wireless communication method is not limited to some specific scheme, and may be any communication scheme, for example, Bluetooth (trade name) or Near Field Communication (NFC).

Furthermore, the data transmission setting unit 706 sets the user-oriented data and the medical professional assistance data to be transmitted to the server 510 via the communication interface 601, sets an address of the area of the server 510 in which the data is stored, and passes the address data indicating the address to the address presentation unit 708. The address presentation unit 708 displays the address data via the output device 204, or prints the address from the printer via the external interface 207 and presents it to the user and the medical professional. The address of the area of the server 510 in which the data is stored, according to the present embodiment corresponds to the “access-enabling data” of the present invention.

The authentication unit 309 performs mutual authentication with the biometric data measurement device 120 that transmits the biometric data to the data generation device 500 via the communication interface 601, and upon authenticating that the biometric data measurement device 120 is the device from which the data generation device 500 is supposed to acquire the biometric data, enables the data generation device 500 to receive the biometric data from the biometric data measurement device 120. In addition, the authentication unit 309 may perform mutual authentication with the server 510 which is the counterpart of transmitting user-oriented data and/or the limited medical professional assistance data or the medical professional assistance data and, upon authenticating that the server 510 is the device to which the biometric data is supposed to be transmitted from the data generation device 500, enables transmission of the user-oriented data and/or the medical professional assistance data to the authenticated server 510.

Other

Operation of the data generation device 500 will be explained in detail in the operation example described below. Note that, in the present embodiment, the control unit 610 of the data generation device 500 may be realized by a general-purpose CPU. However, a part or all of the above-mentioned operations (functions) may be realized by one or more dedicated processors. In addition, components of the data generation device 500 may be omitted, substituted, or added as appropriate in accordance with embodiments.

### Operation Example: Overall

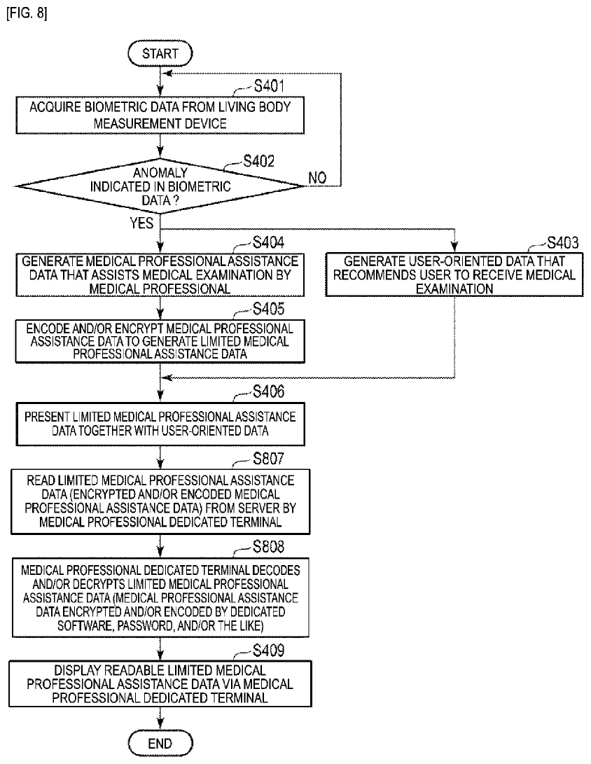

Next, there will be described an outline of an operation of the data generation device 500, referring to FIG. 8.

FIG. 8 is a flowchart illustrating an example of a processing procedure (data generation method) of the data generation device 500. Note that the processing procedure described below is merely an example, and each process may be changed to the extent possible. Further, in the processing procedure described below, steps can be omitted, substituted, and added in accordance with the embodiment as appropriate. Note that, although steps S807, S808 and S409 are not the processing procedure by the data generation device 500, but are the processing procedure by the medical professional terminal 110 described for reference.

Activation

First, the user activates, via the input device 203, the data generation device 500, which in turn accepts input such as settings. The control unit 610 of the data generation device 500 performs the process according to the following processing procedure.

(Step S401)

At step S401, the control unit 610, operating as the biometric data acquisition unit 301, acquires, via the communication interface 601, biometric data of the user from the biometric data measurement device 120 authenticated by the authentication unit 309. The biometric data may be acquired by the biometric data acquisition unit 301, and stored in the storage unit 602 from the biometric data acquisition unit 301 in sequence. The biometric data may be stored in the storage unit 602 in accordance with the date-and-time data of the clocking device 205, or the biometric data may be stored in the storage unit 602 together with the date-and-time data provided by a clocking device of the biometric data measurement device 120 that has acquired the biometric data.

(Step S402)

At step S402, the control unit 610, operating as the biometric data determination unit 302, determines whether or not the biometric data acquired at step S401 indicates an anomaly. The timing of determination generally differs depending on the type of biometric data or the measurement method. For example, in a case where the biometric data is electrocardiographic data, which may vary in seconds, although it is desirable to determine whether or not biometric data indicates anomaly at an interval of seconds or tens of seconds, determination may be performed at a time interval which is acceptable by the acquisition scheme and/or measurement method in a case where the measurement device collectively acquires measured biometric data after completion of measurement, or in a case where it is difficult to measure electrocardiographic data continuously in time (e.g., in seconds). In addition, with the biometric data measurement device 120 starting measurement upon instruction by the user to start measurement, the biometric data acquisition unit 301 may acquire biometric data of the user simultaneously with completion of measurement or start of measurement, and the biometric data determination unit 302 determines whether or not the biometric data indicates an anomaly.

In a case where the biometric data does not indicate any anomaly at step S402, the process returns to step S401, at which the biometric data acquisition unit 301 acquires the biometric data. As long as there exists yet-to-be-determined biometric data that can be determined by the biometric data determination unit 302, the process returns to step S401 to repeat determination.

(Step S403)

At step S403, the control unit 610, operating as the user-oriented data generation unit 303, generates user-oriented data that recommends the user to receive medical examination by a medical professional, because the biometric data indicates an anomaly.

(Step S404)

At step S404, the control unit 610, operating as the medical professional assistance data generation unit 304, generates medical professional assistance data that assists the medical examination performed by the medical professional on the user, because the biometric data indicates an anomaly. Specifically, in a case where there is a symptom indicating arrhythmia in a part of the electrocardiographic data in a single measurement (e.g., 30 seconds), the medical professional assistance data may include raw data, a marker applied to a part (time point) of the raw data indicating possibility of arrhythmia, type of arrhythmia and name of disease that can be estimated therefrom, or examination recommended to be subsequently performed.

(Step S405)

At step S405, the control unit 610, operating as the encryption unit 305, encodes and/or encrypts the medical professional assistance data to generate the limited medical professional assistance data.

(Step S406)

At step S406, the control unit 610, operating as the data transmission setting unit 706 and the data presentation unit 307, displays the encoded and/or encrypted medical professional assistance data (i.e., limited medical professional assistance data) and the user-oriented data on the output device 204, or prints them.

The steps S807, S808 and S409 are not processes performed by the data generation device 500, but are processing procedures by the medical professional terminal 110, for example.

Step S807

At step S807, the medical professional terminal 110 reads the encrypted and/or encoded medical professional assistance data from the server 510 based on the limited medical professional assistance data presented at step S406.

Step S808

At step S808, the medical professional terminal 110 decodes and/or decrypts the encrypted and/or encoded medical professional assistance data using dedicated software and/or a password for decrypting the encrypted and/or encoded medical professional assistance data, and converts the content of the medical professional assistance data into a format that can be read by a medical professional.

(Step S409)

At step S409, the medical professional terminal 110 presents, on a display or the like, the medical professional assistance data converted from the limited medical professional assistance data at step S808 to be knowable by the medical professional.

### Actions and Effects

As has been described above, the data generation device 500 of the present embodiment generates user-oriented data and medical professional assistance data for a medical professional at steps S403 and S404, in a case where the biometric data determination unit 302 has determined that the biometric data indicates an anomaly at step S402. Subsequently, at step S405, the medical professional assistance data is encrypted (or simply encoded) so that only a medical professional can know the content thereof. As a result, the data generation device 500 can generate medical professional assistance data including medical expertise for assisting the medical professional to apply an appropriate medical treatment to the user, and set the medical professional assistance data so that anyone other than a medical professional cannot view it, in other words, it is possible to control to limit the knowability of anyone other than a medical professional.

Furthermore, the data generation device 500 can enable the user to recognize, with the user-oriented data, the necessity to receive medical examination and/or medical practice by a medical professional by presenting the medical professional assistance data together with the user-oriented data to the user at step S406, and, with the medical professional assistance data being presented at the same time, it can be expected that appropriate medical examination and medical practice can be received by passing the data to the medical professional. In addition, from the viewpoint of the medical professional, it can be assumed that the medical professional assistance data that can be read by the medical professional terminal 110 at steps S807, S808, and S409 allows for grasping in detail the reason for receiving medical consultation, which has conventionally been acquired from the user by medical interview or the like, and also it becomes likely to quickly find the cause of the anomaly indicated by the biometric data. In addition, it becomes possible for the user to visit the medical institution within a short time since the biometric data has indicated an anomaly, and therefore the user can be applied medical examination and treatment at an early stage, thereby increasing the possibility of treating the user before the user's symptoms proceeds to a serious stage. In addition, it turns out that medical examination and treatment can be applied at an early stage, making it more likely to provide a wider range of selecting treatment methods, and therefore it becomes possible to apply safer and more appropriate medical practice to the user.

### Third Embodiment

A data generation system 900 of a third embodiment includes the biometric data measurement device 120 and the data generation device 100 of the first embodiment. The biometric data measurement device 120 is, for example, an electrocardiograph, a sphygmomanometer, a heart rate meter, a pedometer, and an activity meter. An example of a hardware configuration of the biometric data measurement device 120 includes a biometric data measurement unit 1201 configured to perform measurement of biometric data, a storage unit 1202, a communication interface 1203, and a control unit 1210, although details thereof are omitted. The biometric data measurement unit 1201, which performs measurement of biometric data, is configured in various manners according to the biometric data measured. The storage unit 1202 has stored therein measured biometric data, and an execution program for measuring biometric data and transmitting the measured data to the data generation device 100. The communication interface 1203 is an interface for performing wired or wireless communication via a network, for example, a near-field wireless communication (e.g., Bluetooth (trade name)) module, a wired Local Area Network (LAN) module, or a wireless LAN module. The communication interface 1203 is an interface for connecting the biometric data measurement device 120 to an external device such as the data generation device 100. In addition, the storage unit 1202 may include a portable storage medium such as a memory card or a USB memory.

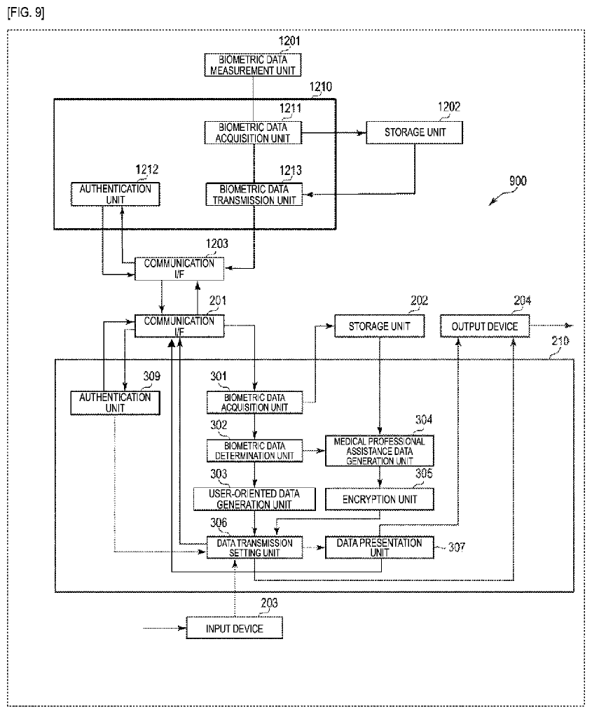

FIG. 9 illustrates an example of a software configuration of a data generation system 900. The data generation device 100 is similar to that explained for the first embodiment, and therefore description thereof will be omitted. The biometric data measurement device 120 indicates a software configuration for executing a program to be executed by the control unit 1210 for measuring biometric data and transmitting the biometric data to the data generation device 100.

The biometric data acquisition unit 1211 acquires the biometric data output from the biometric data measurement unit 1201, and stores it in the storage unit 1202.

The biometric data transmission unit transmits, to the data generation device 100 via the communication interface 1203, the biometric data stored in the storage unit 1202 or the biometric data output from the biometric data acquisition unit 1211.

The authentication unit 1212 performs mutual authentication with the data generation device 100 that transmits the biometric data via the communication interface 201, and upon authenticating that the data generation device 100 is the device to which the biometric data measurement device 120 is supposed to transmit the biometric data, enables transmission of the biometric data from the biometric data measurement device 120 to the data generation device 100.

The data generation system 900 configured as described above has similar functions and effects to those of the data generation device 100 of the first embodiment. In the above description, there is also conceivable a configuration including the biometric data measurement device 120 and the data generation device 500 of the second embodiment.

### Fourth Embodiment

A data generation device 1000 of a fourth embodiment is configured with the biometric data measurement device 120 being integrated into the data generation device 100. In the description of the fourth embodiment, similar reference numerals are used for components similar to those of the first embodiment, and only the difference from the first embodiment will be described.

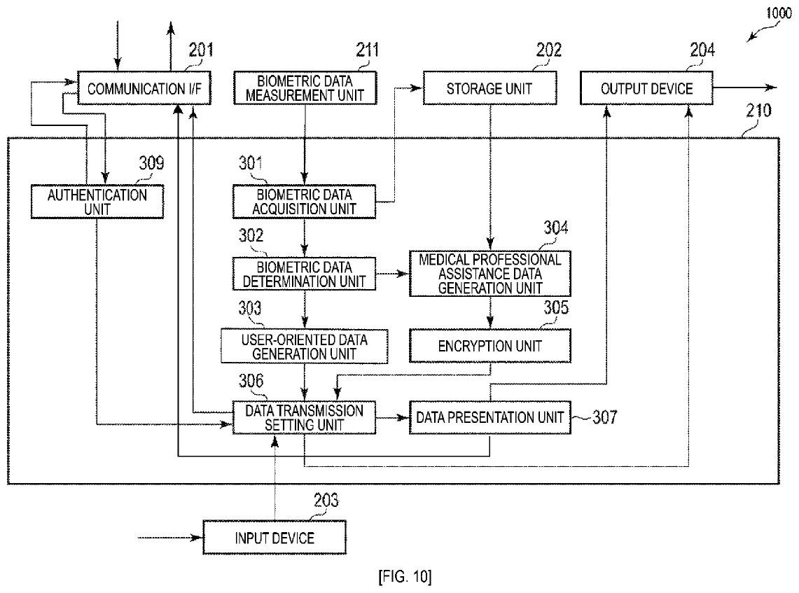

The hardware configuration of the data generation device 1000 has the biometric data measurement unit 211 added to the hardware configuration of the data generation device 100 illustrated in FIG. 2. The biometric data measurement unit 211, which performs measurement of the biometric data, is configured in various manners according to the biometric data measured. In the following, there will be described a software configuration of the measurement device 1000, referring to FIG. 10.

Here, the data output from the biometric data measuring unit 211 is acquired by the biometric data acquisition unit 301. In a case where the biometric data is electrocardiographic data, the biometric data acquisition unit 301 acquires, as the electrocardiographic data, a potential difference signal indicating a potential difference between two electrodes output in time series from the signal processing circuit in the biometric data measurement device 211. Here, the electrocardiographic data is a waveform signal representing electrical activity of the heart. In a case where the biometric data is blood pressure data, the biometric data acquisition unit 301 acquires blood pressure data measured by the biometric data measurement unit 211.

For example, in a case where the data generation device 1000 is a wearable terminal device (e.g., smartphone), it usually includes an acceleration sensor and a pressure sensor so that it can measure the number of steps and stairs, and therefore it is possible to calculate biometric data based on the number of steps and stairs.

As has been described above, forming the data generation device 1000 integrally with the biometric data device allows a single device to perform measurement of the biometric data and generation of the user-oriented data and the medical professional assistance data, and therefore an easy-to-use and highly portable data generation device can be realized.

### Modified Examples

While embodiments of the present invention have been described in detail above, the foregoing description is merely illustrative of the present invention in all respects. Of course, various modifications and variations can be made without departing from the scope of the present invention. For example, the following changes are possible. Further, specific configurations according to the embodiment may be adopted as appropriate in the implementation of the invention. Note that, in the following, the same reference numerals are used for components that are the same as those of the above-described embodiment, and descriptions thereof are omitted as appropriate. The following modification examples may be combined as appropriate.

<1>

The data transmission setting unit 306 may be configured to present, to the output device 204, a password for decrypting the encrypted medical professional assistance data. The medical professional can decrypt the medical professional assistance data encrypted using the presented password. Note that, in the present case, the encrypted medical professional assistance data assumes usage of a system such that the encrypted medical professional assistance data cannot be decrypted by the user or a third party using only the address of the server having stored the medical professional assistance data therein and the password for decryption, but requires a combination of a system and/or a medical professional ID and the like possessed by only the medical professional for decrypting the encrypted medical professional assistance data.

<2>

In the data generation device 100 of the first embodiment and the data generation device 500 of the second embodiment described above, the user-oriented data and the medical professional assistance data are generated, triggered by the biometric data indicating an anomaly. However, in a case where the user has a chronic disease, the data generation device may generate user-oriented data and medical professional assistance data, triggered by acquisition of a predetermined amount (number of times) of the biometric data or an elapse of a predetermined time period after having acquired biometric data continuously, even when the biometric data is not indicating an anomaly. On this occasion, the data generation device 100 illustrated in FIG. 2 or the data generation device 500 illustrated in FIG. 6 corresponds to the “data generation device” of the present invention, as the hardware configuration. However, the biometric data determination unit 302 illustrated in FIGS. 3 and 7 can be omitted in the data generation device of the present invention as the software configuration. The biometric data determination unit 302 may be provided to operate like the data generation device 100 of the first embodiment or the data generation device 500 of the second embodiment in a case where an anomaly has occurred, and operate like the present modification in a case where there is no anomaly. Here, in place of determining whether or not the acquired biometric data indicates an anomaly at step S402 of the flowchart illustrated in FIGS. 4 and 8, the control units 210 and 610 determine whether or not a predetermined amount (number of times) of biometric data has been acquired or whether or not the acquisition time period of the biometric data has exceeded a predetermined time period. Subsequently, in a case where such a condition is satisfied, the user-oriented data and the generation of medical professional assistance data are generated, in a case where the condition is not satisfied, the process returns to step S401 at which the biometric data acquisition unit 301 acquires the biometric data.

Here, the doctor may set the amount (number of times) or time period that triggers the generation of data in the input device 203 or from the medical professional terminal, or may instruct the user. In addition, in a case where the user periodically receives medical examination by the doctor, it is conceivable to store the scheduled date of the next medical consultation and generate the user-oriented data to guide the next medical consultation when the scheduled date of medical consultation is coming up. In such a case, it is preferred that the medical professional assistance data includes the biometric data acquired for the above-described predetermined amount (number of times) or a predetermined time period, and further includes biometric data, user-related usage data, and lifestyle data until receiving medical examination by the doctor after having been acquired for a predetermined amount (number of times) or a predetermined time period. Additionally, in such a case, there may be generated, as the user-oriented data, data indicating that there is no anomaly, data indicating recommendation for continuing observation of biometric data, or data prompting periodic reception of medical consultation.

<3>

The data generation device 500, the medical professional terminal 110, and the server 510 in the second embodiment can also form a part of the system for the case where the user receives online medical practice by a medical professional. In the foregoing manner, the user is relieved from the trouble of visiting the medical professional, and the medical professional can quickly perform medical examination using the useful medical professional assistance data without having to wait for the user.

<4>

The first and the second embodiments limit the user's knowability about the content of medical professional assistance data generated based on the user's biometric data. As has been described above, the biometric data for the user is related to the user's privacy, and therefore its protection is required. Such protection is confidentiality against a third party, which is realized by a component corresponding to the access control unit of the present invention. Furthermore, in recent years, self-information control right has been drawing attention as an aspect of privacy protection. The foregoing refers to a right that anyone can control, by him/herself, handling of information related to him/herself. Although the data included in the medical professional assistance data has been generated based on the biometric data of the user, the data need not be known by the user, or the data may be inappropriate and content of the data need not be known. However, from the perspective of the self-information control rights, it is preferred that the access control unit controls the content of the data included in the medical professional assistance data to be knowable, as long as the user desires. In such a case, a control is performed to mitigate the limitation so that the limitation on knowability about content of the medical professional assistance data is minimized. For example, it is conceivable to display an interface for displaying or the like the user-oriented data and an interface for displaying or the like the medical professional assistance data separately on the display unit.

In addition, the user may be allowed to set the knowability about the content of medical professional assistance data generated from the user's biometric data.

<5>

Although not illustrated, the biometric data measurement device 120 and/or the data generation device 500 may include an acceleration sensor, a pressure sensor, a gyro sensor, and/or a geomagnetic sensor.

The acceleration sensor, which is a sensor for detecting acceleration for example a three-axis acceleration sensor, detects the acceleration of the sensor with respect to three linearly independent axes (e.g., three mutually orthogonal axes). The acceleration sensor then outputs an acceleration signal indicating acceleration in three directions to the control unit 210. The acceleration sensor can acquire a roll angle and a pitch angle from the value of acceleration in stationary state.

The pressure sensor is of a general type that detects pressure, by using the pressure sensor, it is possible to detect the user's altitude by measuring the atmospheric pressure, for example.

The gyro sensor is of a general type that can detect angular velocity of a sensor, for example, a three-axis gyro sensor that detects the angular velocity of the sensor with respect to three linearly independent axes. The gyro sensor outputs an angular velocity signal representing angular velocity in three directions to the control unit 210.

The geomagnetic sensor is a general type and used to determine the posture of the user. The geomagnetic sensor, which is a 3-axis geomagnetic sensor, for example, detects the intensity of the geomagnetic field around the sensor including direction and strength (magnitude) with respect to three axes.

Using a method of correcting an error, due to drift, of the angular velocity acquired from the gyro sensor, the control unit 210 may acquire, from information of the acceleration sensor and the geomagnetic sensor, an angle by integrating the angular velocity from the initial posture information of the user, and acquire a posture angle for each sensor at a desired time elapsed since the initial time. As the initial posture, by the acceleration sensor, the roll angle and the pitch angle can be acquired. The yaw angle can be determined using the geomagnetic sensor.

The control unit 210 calculates three-dimensional components of the magnetic field with corrected tilt error, from the three-dimensional components of the magnetic field acquired by the geomagnetic sensor and the roll angle and the pitch angle preliminarily acquired. The yaw angle can be calculated from the x component and the y component among the three-dimensional components of the magnetic field with corrected tilt error. For example, when the user moves from the initial posture, an angle is acquired by time-integrating the angular velocity, whereby it is possible to acquire a posture angle for each sensor at an arbitrary time point from the initial posture and the elapsed time.

<6>

The device of the present invention may also be realized by a computer and a program, and the program may be recorded in a recording medium (or storage medium), or provided through a network.

Further, the above-described devices and the device portions thereof can each be implemented by either a hardware configuration or a combined configuration of a hardware resource and software. As software configured by combination, a program is used, which has been preliminarily installed in a computer from a network or a computer-readable recording medium (or storage medium) and which, when executed by a processor of the computer, causes the computer to realize the operation (function) of each device.

<6>

Note that this invention is not limited to the embodiments described above, and can be embodied by modifying the components in an implementation stage without departing from the gist thereof. Additionally, various inventions can be formed by appropriately combining a plurality of components disclosed in the embodiments described above. For example, some components may be omitted from all the components described in the embodiments. Further, the components of the different embodiments may be combined appropriately.

<7>

Further, “and/or” means any one or more items of the items connected and listed by the “and/or” statement. As a specific example, “x and/or y” means any element of the set of three elements {(x), (y), (x, y)}. As another specific example, “x, y, and/or z” means any element of the set of seven elements {(x), (y), (z), (x, y), (x, z), (y, z), (x, y, z)}.

Supplementary Note 1

A data generation device (100, 500) including, a biometric data acquisition unit (301) configured to acquire biometric data of a user, a generation unit (303, 304, 305) configured to generate, based on the biometric data, user-oriented data and medical professional assistance data assisting an activity performed by a medical professional in relation to the user, an access control unit (304, 305, 306, 309, 706) configured to control knowability about the content of the medical professional assistance data, and a data presentation unit (307) configured to present the user-oriented data to the user.

### REFERENCE SIGNS LIST

100, 500, 1000 Data generation device
110 Doctor terminal
120 Wristwatch-type wearable terminal
201, 601 Communication interface
202, 602 Storage unit
203 Input device
204 Output device
205 Clocking device
206 Power source unit
207 External interface
210, 610 Control unit
301 Biometric data acquisition unit
302 Biometric data determination unit
303 User-oriented data generation unit
304 Medical professional assistance data generation unit
305 Encryption unit
306, 706 Data transmission setting unit
307 Data presentation unit
309 Authentication unit
510 Server
520 Network
708 Address presentation unit
900 Data generation system

1. A data generation device comprising:
a memory; and
a processor configured to:
acquire biometric data of a user;
generate, based on the biometric data, user-oriented data directed to the user, and medical professional assistance data directed to a medical professional, the medical professional assistance data assisting an activity performed by the medical professional in relation to the user;
control knowability about content of the medical professional assistance data; and
present the user-oriented data to the user, wherein
the processor generates access-enabling data that releases the limitation on the knowability or enhances the knowability, to a specified medical professional, about the content of the medical professional assistance data, and
the processor displays, or transmits an instruction for printing, the access-enabling data together with the user-oriented data.

2. The data generation device according to claim 1, wherein the access-enabling data is a password to be presented to the specified medical professional to release the limitation on the knowability, to the specified medical professional, about the content of the medical professional assistance data.

3. The data generation device according to claim 1, further comprising a transmitter configured to transmit the medical professional assistance data to a server, wherein
the processor sets an area in the server, where the medical professional assistance data is stored, to have a limitation on the knowability about the content of the medical professional assistance data stored in the area.

4. The data generation device according to claim 3, wherein
the processor generates an address of a server having the medical professional assistance data stored therein, as access-enabling data for releasing the limitation on the knowability or enhancing the knowability, to the specified medical professional, about the content of the medical professional assistance data, and
the processor displays, or transmits an instruction for printing, the address together with the user-oriented data.

5. The data generation device according to claim 1, wherein the processor is further configured to specify an access destination, having the medical professional assistance data stored therein, as access-enabling data for enhancing the knowability.

6. The data generation device according to claim 1, wherein the processor generates limited medical professional assistance data having limited knowability about the content of the medical professional assistance data.

7. The data generation device according to claim 6, wherein the processor generates the medical professional assistance data as limited medical professional assistance data in a form of data described in a two-dimensional code.

8. The data generation device according to claim 1, wherein the processor displays, or transmits an instruction for printing, the medical professional assistance data together with the user-oriented data.

9. The data generation device according to claim 1, further comprising a display configured to display an interface that enables the user to execute a process of transmitting at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

10. The data generation device according to claim 1, wherein
the processor is further configured to store, in a portable storage medium, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, and
the processor presents user-oriented data indicating that at least one of the data is stored in the storage medium, and prompting to pass the storage medium to the medical professional.

11. The data generation device according to claim 1, wherein
the processor is further configured to set at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data to be transmittable to the terminal of the medical professional, and
the processor presents user-oriented data that guides the user to transmit, to the terminal of the medical professional, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, so that the medical professional can know the medical professional assistance data when the user receives an activity related to the user from the medical professional.

12. The data generation device according to claim 1, further comprising a transmitter configured to transmit, according to the near-field wireless communication method, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, wherein
the processor, upon authenticating that the transmission destination is the terminal of the medical professional, transmits, to the terminal of the medical professional, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

13. The data generation device according to claim 12, further comprising a display configured to display an interface that accepts permission of the user for association with the terminal of the medical professional, wherein
the processor, upon accepting a request from the terminal of the medical professional and authenticating that the request is from the terminal of the medical professional associated based on the permission, transmits, to the terminal of the medical professional, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

14. The data generation device according to claim 1, wherein
the processor presents user-oriented data that guides to display or transmit an instruction for printing, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data, when the user receives, from a medical professional, an activity performed by the medical professional in relation to the user, and
the processor displays, or transmits an instruction for printing, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

15. The data generation device according to claim 14, further comprising a display configured to display an interface that accepts permission of the user for association with the terminal of the medical professional, wherein
the processor, upon accepting a request from the terminal of the medical professional and authenticating that the request is from the terminal of the medical professional associated based on the permission, displays, or transmits an instruction for printing, at least one of the medical professional assistance data, limited medical professional assistance data having limited knowability about the content of the medical professional assistance data, and access-enabling data for releasing the limitation on the knowability or enhancing the knowability, to a specified medical professional, about the content of the medical professional assistance data.

16. The data generation device according to claim 1, wherein
the processor is further configured to determine whether or not the biometric data indicates an anomaly, and
in a case where it has been determined that the biometric data indicates an anomaly, the user-oriented data includes a message that recommends the user to receive the activity from the medical professional.

17. The data generation device according to claim 16, wherein the processor generates, by incorporating into the medical professional assistance data, at least one of biometric data of the user acquired from after the determination to when the user receives the activity from the medical professional, medical data related to the user, and lifestyle data.

18. The data generation device according to claim 17, wherein the processor generates, by incorporating into the medical professional assistance data, in accordance with the determination, at least one of biometric data of the user prior to the determination, medical data related to the user, and lifestyle data, the three sets of data having been preliminarily set.

19. The data generation device according to claim 16, wherein the processor further presents to prompt the user to inform the medical professional of existence of medical professional assistance data, or pass the medical professional assistance data to the medical professional.

20. The data generation device according to claim 16, wherein the processor determines a level indicating the degree of the anomaly based on the biometric data, and determines that the biometric data is indicating an anomaly in a case where the level is equal to or higher than a threshold value.

21. The data generation device according to claim 16, wherein the medical professional assistance data includes anomaly-specifying data for specifying data related to an anomaly in the acquired biometric data.

22. A data generation method causing a computer to execute:
acquiring biometric data of a user;
generating, based on the biometric data, user-oriented data directed to the user, and medical professional assistance data directed to a medical professional, the medical professional assistance data assisting an activity performed by the medical professional in relation to the user;
generating access-enabling data that releases the limitation on the knowability or enhances the knowability about the content of the specified medical professional assistance data; and
displaying, or transmitting an instruction for printing, the access-enabling data together with the user-oriented data.

23. A non-transitory storage medium storing a program for causing a computer to function as the data generation device according to claim 1.
